(19) United States
(12) Patent Application Publication
(10) Pub. No.: US 2024/0100083 A1
(43) Pub. Date: Mar. 28, 2024

(54) IMMUNOTHERAPY FOR CANCER

(71) Applicant: THE PROVOST, FELLOWS, FOUNDATION SCHOLARS, AND THE OTHER MEMBERS OF BOARD, OF THE COLLEGE OF THE HOL, Dublin 2 (IE)

(72) Inventors: Ed LAVELLE, Dublin 2 (IE); Joanna TURLEY, Dublin 2 (IE); Ross WARD, Dublin 2 (IE)

(21) Appl. No.: 18/039,107

(22) PCT Filed: Nov. 26, 2021
(86) PCT No.: PCT/IB2021/061020

§ 371 Date: May 26, 2023

(30) Foreign Application Priority Data
Nov. 26, 2020 (GB) 2018665.6

(51) Int. Cl. A61K 31/722; A61K 9/00; A61K 45/06; A61P 35/00

(52) U.S. Cl. CPC A61K 31/722; A61K 9/0019; A61K 45/06; A61P 35/00

(57) ABSTRACT

The invention relates to a method of immunotherapy for prevention or treatment of cancer in a subject, the method comprising the administration of an adjuvant, or administration of an adjuvant in combination with a tumour antigen, to the subject, wherein the adjuvant comprises polyglucosamine or acetylated polyglucosamine that comprises no more than 10% N-acetyl-D-glucosamine groups, and wherein the N-acetyl-D-glucosamine groups are distributed in the acetylated polyglucosamine in blocks of two or more. The invention relates to a method of immunotherapy for solid-tumour cancer in a subject, the method comprising the administration of polyglucosamine or chitosan into the subject, wherein the administration is intratumoral and/or peritumoral.

This invention relates to immunotherapy for cancer, particularly the use of an adjuvant for anti-tumour vaccines, and related methods and compositions. The invention further relates to anti-cancer therapeutics and related methods and compositions.

Cancer immunotherapy has emerged as a new pillar of cancer treatment, and research in the area has exploded in the past decade, leading to the development of several blockbuster drugs. Cancer vaccines will be the next frontier in this new age of cancer therapy; however, their development has been hampered by lack of suitable vaccine adjuvants, components of a vaccine which aid in the initiation and propagation of protective immune responses. Vaccines could have applications in preventing cancer (prophylactically) or in treatment of existing tumours (therapeutic). A vaccine adjuvant that could be used in both settings would have a stellar impact and a wide breadth of applications.

Harnessing innate immunity to treat cancer is at the cutting edge of targeted and personalised cancer treatment. Recent studies reveal critical roles for cGAS-STING dependent innate immune signalling in natural anti-tumour immunity, leading to great interest in developing activators of this pathway as cancer therapeutics. To date, research has focused on cyclic dinucleotide (CDN) analogues. These molecules are synthesised by the enzyme, cGAS after detection of DNA within a cell. CDNs activate STING, which leads to the expression of potent anti-viral and anti-cancer cytokines that trigger adaptive immunity. Indeed, intratumoral injections of CDNs have led to clearance of multiple tumour types in mice and evaluation in phase I clinical trials. Unfortunately, these CDN STING agonists show less efficacy in humans and have halted at phase Ib phases. Their failure is thought to be due to their inability to enter cells and activate variable forms of the STING protein across human populations. As such, the search continues to find a translational STING agonist.

Therefore, the aim of the invention is to provide an improved vaccine adjuvant for cGAS-STING pathway activation and/or alternative cancer therapies.

According to a first aspect of the present invention, there is provided a method of immunotherapy for prevention or treatment of cancer in a subject, the method comprising the administration of an adjuvant, or administration of an adjuvant in combination with a tumour antigen, to the subject, wherein the adjuvant comprises polyglucosamine or acetylated polyglucosamine that comprises no more than 10% N-acetyl-D-glucosamine groups, and wherein the N-acetyl-D-glucosamine groups are distributed in the acetylated polyglucosamine in blocks of two or more.

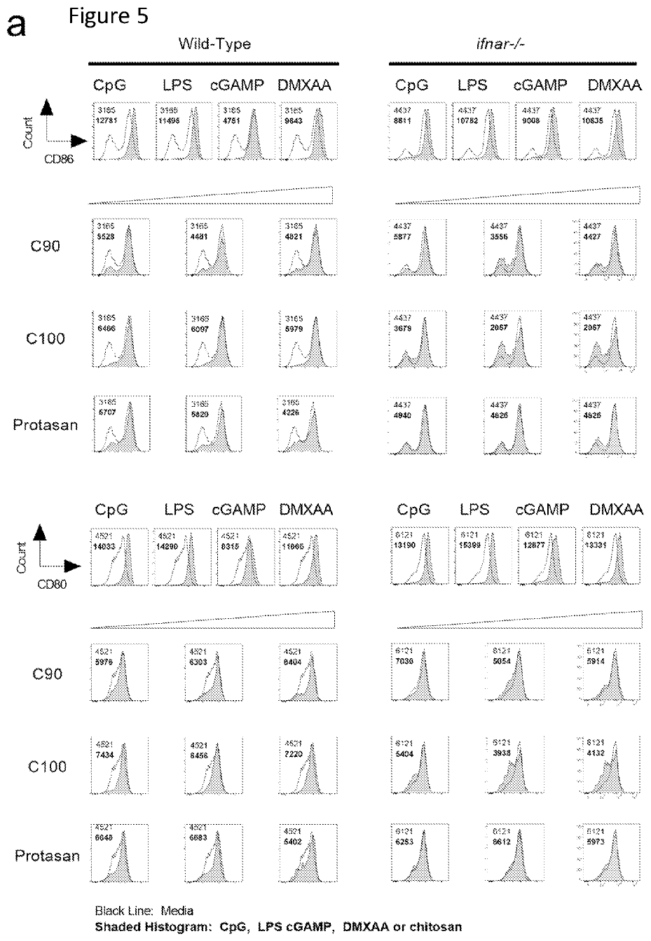

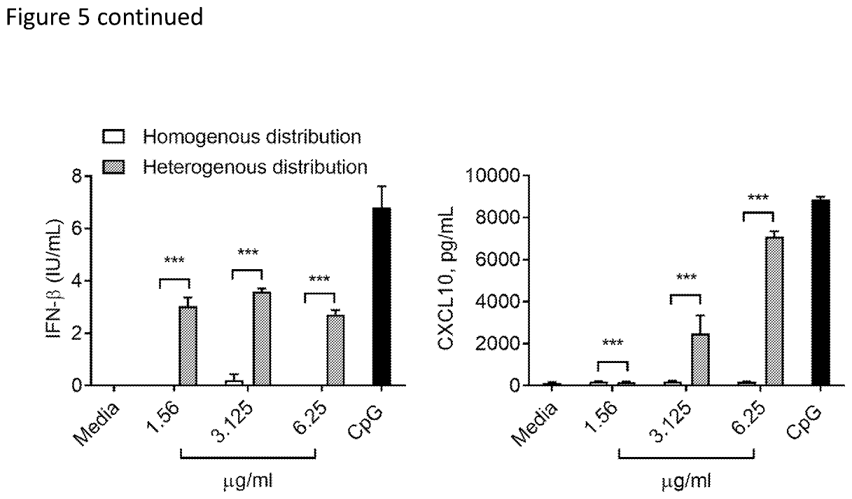

The invention advantageously provides an adjuvant with unique potential to promote anti-tumour immune responses. It has been found that the carbohydrate polyglucosamine (a 100% de-acetylated form of chitin) readily enters cells and efficiently activates cGAS-STING, possessing impressive anti-tumour functionality in multiple mouse pre-clinical models in prophylactic and therapeutic settings. The invention advantageously displays anti-tumour and STING activity in human monocytic leukaemia cell line (THP1). Vaccination approaches aiming to target intracellular pathogens and cancer require components of cellular immunity to be activated. Currently there is a lack of suitable adjuvants to elicit such responses. While commercially available chitosan salts have been shown to promote cellular immune responses in vivo following vaccination via the intraperitoneal and intranasal route, these have more limited ability to promote such responses by clinically relevant routes (such as intramuscularly or subcutaneously). In particular Carroll et al. (2016, Immunity 44, 597-608) has previously used Protasan™ (commercially available chitosan with an 86% degree of deacetylation (DD). It was understood that chitosan critically relies on the induction of mitochondrial reactive oxygen species (mtROS) to drive mtDNA release, cGAS activation, cGAMP production and STING activation. With 90-100% deacetylation of the chitin, the levels of mtROS are surprisingly elevated, and the levels are significantly higher with polyglucosamine (100% deacetylated form of chitin). Activation of dendritic cells by polyglucosamine is via a different mechanism where the polymer appears to damage the nuclear compartment, allowing the release of nuclear DNA into the cytosol and activating the cGAS-STING pathway. Polyglucosamine and chitosan of higher de-acetylation of at least 90% has potent anti-tumour functionality in prophylactic pre-clinical animal tumour models, significantly outperforming Protasan™ (FIG. 5). The anti-tumour functionality of induced responses in a prophylactic setting using robust pre-clinical animal tumour models has been demonstrated. The mode of action of polyglucosamine has been elucidated and this provides a clear rationale for the use of polyglucosamine and acetylated polyglucosamine, such as chitosan with de-acetylation of at least 90% as a vaccine adjuvant, as a clear understanding of a compounds mechanism of action is important for translation into clinical settings. The adjuvant of the present invention also differs from those discussed in the art, such as WO2020/045679 as they are advantageously free chains, and not in clumps, because unlike the molecules of WO2020/045679, the free amino group may not be attached to anything such as surfactant or TLR agonist.

According to a second aspect of the present invention, there is provided a method of immunotherapy for solid-tumour cancer in a subject, the method comprising the administration of polyglucosamine or chitosan into the subject, wherein the administration is intratumoral and/or peritumoral.

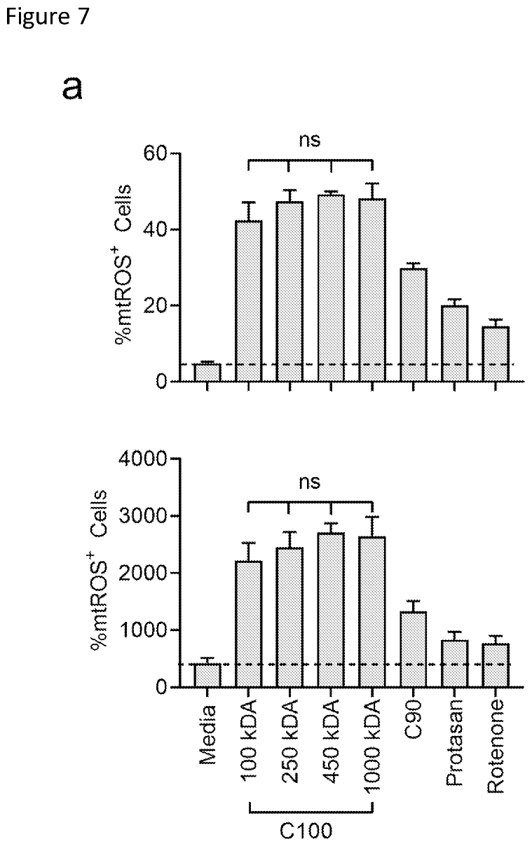

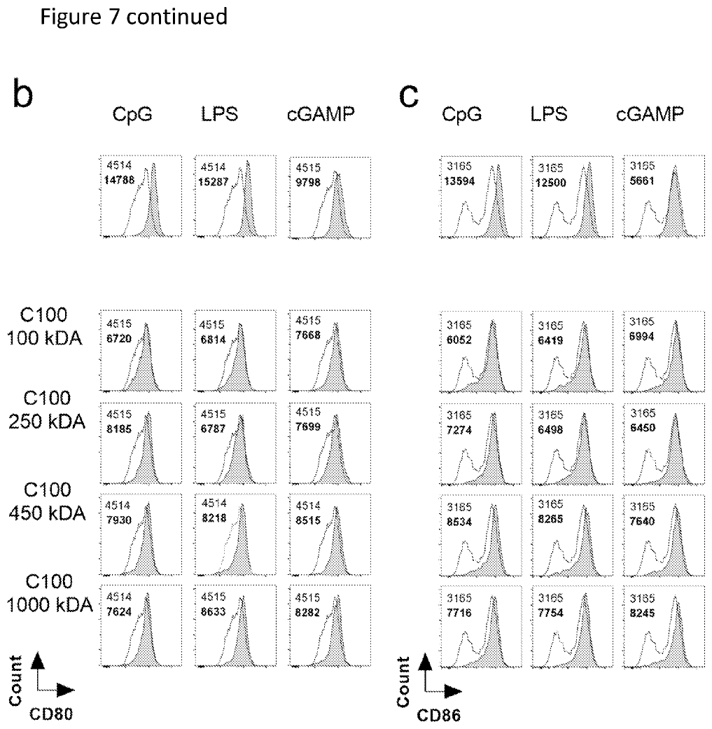

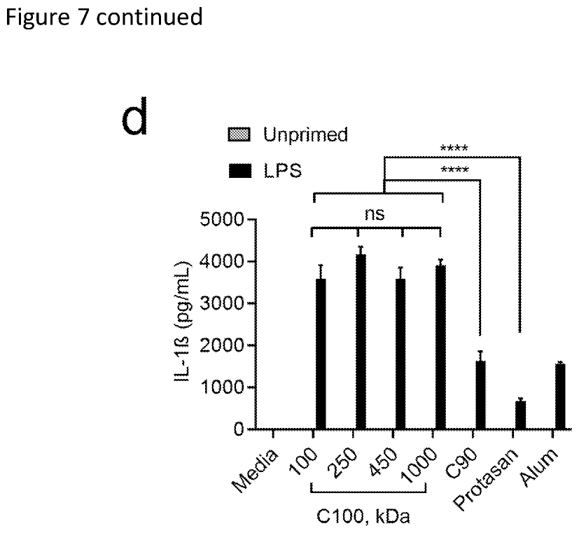

Advantageously the invention further identifies that polyglucosamine (also chitosan) is an effective standalone cancer immunotherapeutic. It is demonstrated herein that intratumoral injections of polyglucosamine (or chitosan) results in significant inhibition of tumour growth in pre-clinical animal tumour models. Intratumoral polyglucosamine (or chitosan) injection can be used in standalone and combinatorial approaches, with significant potential for synergy alongside checkpoint inhibitor and costimulatory agonist therapy. The presence of intratumoral immune cells is a prerequisite for successful response to checkpoint (coinhibitory receptor) blockade therapies. Intratumoral polyglucosamine (or chitosan) delivery likely leads to an influx of immune cells, transitioning tumours from immunologically ‘cold’ to ‘hot’, and STING agonists like polyglucosamine have been shown to upregulate checkpoint expression on immune cells. It is also shown herein that polyglucosamine (or chitosan) can upregulate costimulatory molecule expression on tumour cells themselves (FIG. 7). Furthermore, it has been demonstrated that polyglucosamine (or chitosan) can have direct tumoricidal activity. Thus, there is a significant capacity for polyglucosamine (or chitosan) to enhance response rates to checkpoint inhibitor treatments, which critically remain low clinically.

The Chitosan and Polyglucosamine

“Chitosan” is a linear polysaccharide composed of randomly distributed β-(1→4)-linked D-glucosamine (deacetylated unit) and N-acetyl-D-glucosamine (acetylated unit). Chitosan is typically produced commercially by deacetylation of chitin, which is the structural element in the exoskeleton of crustaceans (such as crabs and shrimp) and cell walls of fungi. Chitin has at least 90% acetylation. The degree of deacetylation (% DD) of chitosan refers to the percentage of deacetylated units (β-(1→4)-linked D-glucosamine) in the molecule, and it may be determined by NMR spectroscopy. Chitosan may otherwise be referred to as “acetylated polyglucosamine”. In one embodiment, the chitosan (acetylated polyglucosamine) is derived from a synthetic (non-natural) source, such as from linking monomeric glucosamines and/or monomeric N-acetyl-D-glucosamines.

“Polyglucosamine” herein refers to a polymer of β-(1→4)-linked D-glucosamine that does not comprise N-acetyl-D-glucosamine. The term “polyglucosamine” may be used interchangeably with “100% de-acetylated chitin” (i.e. chitosan polymer that is free of acetyl groups).

“Acetylated polyglucosamine” herein refers to a polymer of β-(1→4)-linked D-glucosamine that additionally comprises N-acetyl-D-glucosamine groups. The term “acetylated polyglucosamine” may be used interchangeably with “chitosan”. The acetylated polyglucosamine may not be chitin derived. For example the acetylated polyglucosamine or polyglucosoamines may be synthetically produced from monomeric subunits thereof, such as monomeric glucosamines and/or monomeric N-acetyl-D-glucosamine. The degree of acetylation (%) of the acetylated polyglucosamine refers to the percentage of acetylated units (β-(1→4)-linked D-glucosamine) in the molecule, and it may be determined by NMR spectroscopy.

The polyglucosamine or acetylated polyglucosamine/chitosan may not comprise glutamate. The acetylated polyglucosamine/chitosan may not be protasan glutamate. The polyglucosamine or acetylated polyglucosamine according to the invention may not form clumps/aggregates. The polyglucosamine or acetylated polyglucosamine according to the invention may not be in the form of particulate. In one embodiment, the polyglucosamine or acetylated polyglucosamine is in free chain (e.g. non-aggregate) form. In one embodiment, the polyglucosamine or acetylated polyglucosamine is positively charged such that is it in chain form, for example in a composition. In one embodiment, the polyglucosamine or acetylated polyglucosamine is a chain with a net positive charge. The polyglucosamine or acetylated polyglucosamine may not be cross-linked or linked to another molecule, such as an antigen or ligand. In one embodiment, the amino group of the polyglucosamine or acetylated polyglucosamine may not be covalently attached to another molecule, such as an antigen or ligand. The polyglucosamine or acetylated polyglucosamine may not be linked to a TLR agonist or a surfactant. Such linking of other molecules such as surfactant or TLR agonist can neutralise the net positive charge and cause unwanted clumping.

In a preferred embodiment, the polyglucosamine or acetylated polyglucosamine is not hydrolyzed.

In one embodiment according to the first aspect, the acetylated polyglucosamine is no more than 8% acetylated. In another embodiment according to the first aspect, the acetylated polyglucosamine is no more than 6% acetylated. In another embodiment according to the first aspect, the acetylated polyglucosamine is no more than 5% acetylated. In another embodiment according to the first aspect, the acetylated polyglucosamine is no more than 4% acetylated. In another embodiment according to the first aspect, the acetylated polyglucosamine is no more than 3% acetylated. In another embodiment according to the first aspect, the acetylated polyglucosamine is no more than 2% acetylated. In another embodiment according to the first aspect, the acetylated polyglucosamine is no more than 1% acetylated. In another embodiment according to the first aspect, the acetylated polyglucosamine is no more than 0.5% acetylated.

In one embodiment according to the first aspect, the acetylated polyglucosamine is chitosan and is at least 90% de-acetylated. In another embodiment according to the first aspect, the acetylated polyglucosamine is chitosan and is at least 92% de-acetylated. In another embodiment according to the first aspect, the acetylated polyglucosamine is chitosan and is at least 95% de-acetylated. In another embodiment according to the first aspect, the acetylated polyglucosamine is chitosan and is at least 98% de-acetylated. In another embodiment according to the first aspect, the acetylated polyglucosamine is chitosan and is at least 99% de-acetylated. In another embodiment according to the first aspect, the acetylated polyglucosamine is chitosan and is 100% de-acetylated.

According to the second aspect, the acetylated polyglucosamine is chitosan and may be at least 10% de-acetylated. In another embodiment according to the second aspect, the acetylated polyglucosamine is chitosan and is at least 15% de-acetylated. In another embodiment according to the second aspect, the acetylated polyglucosamine is chitosan and is at least 20% de-acetylated. In another embodiment according to the second aspect, the acetylated polyglucosamine is chitosan and is at least 38% de-acetylated. In another embodiment according to the second aspect, the acetylated polyglucosamine is chitosan and is at least 40% de-acetylated. In another embodiment according to the second aspect, the acetylated polyglucosamine is chitosan and is at least 49% de-acetylated. In another embodiment according to the second aspect, the acetylated polyglucosamine is chitosan and is at least 50% de-acetylated. In another embodiment according to the second aspect, the acetylated polyglucosamine is chitosan and is at least 70% de-acetylated. In another embodiment according to the second aspect, the acetylated polyglucosamine is chitosan and is at least 72% de-acetylated. In another embodiment according to the second aspect, the acetylated polyglucosamine is chitosan and is at least 80% de-acetylated. In another embodiment according to the second aspect, the acetylated polyglucosamine is chitosan and is at least 90% de-acetylated. In another embodiment according to the second aspect, the acetylated polyglucosamine is chitosan and is at least 95% de-acetylated. In another embodiment according to the second aspect, the acetylated polyglucosamine is chitosan and is at least 98% de-acetylated. In another embodiment according to the second aspect, the acetylated polyglucosamine is chitosan and is at least 99% de-acetylated. In another embodiment according to the second aspect, the polyglucosamine is not acetylated, and is chitosan that is 100% de-acetylated.

In one embodiment according to the second aspect, the acetylated polyglucosamine is no more than 90% acetylated. In another embodiment according to the second aspect, the acetylated polyglucosamine is no more than 80% acetylated. In another embodiment according to the second aspect, the acetylated polyglucosamine is no more than 60% acetylated. In another embodiment according to the second aspect, the acetylated polyglucosamine is no more than 50% acetylated. In another embodiment according to the second aspect, the acetylated polyglucosamine is no more than 30% acetylated. In another embodiment according to the second aspect, the acetylated polyglucosamine is no more than 20% acetylated. In another embodiment according to the second aspect, the acetylated polyglucosamine is no more than 15% acetylated. In another embodiment according to the second aspect, the acetylated polyglucosamine is no more than 10% acetylated. In another embodiment according to the second aspect, the acetylated polyglucosamine is no more than 8% acetylated. In another embodiment according to the second aspect, the acetylated polyglucosamine is no more than 5% acetylated. In another embodiment according to the second aspect, the acetylated polyglucosamine is no more than 2% acetylated. In another embodiment according to the second aspect, the acetylated polyglucosamine is no more than 1% acetylated. In another embodiment according to the second aspect, the acetylated polyglucosamine is no more than 0.5% acetylated.

Preferred is to use polyglucosamine or a chitosan polymer having not less than 90% of its acetyl groups removed. Even more preferred is to use polyglucosamine or a chitosan having less than 1% of its glucosamine units acetylated.

The N-acetyl-D-glucosamine groups may be distributed in the acetylated polyglucosamine in blocks of three or more. The N-acetyl-D-glucosamine groups may be distributed in the acetylated polyglucosamine in blocks of four or more. The N-acetyl-D-glucosamine groups may be distributed in the acetylated polyglucosamine in blocks of five or more. The N-acetyl-D-glucosamine groups may be distributed in the acetylated polyglucosamine in blocks of ten or more.

The N-acetyl-D-glucosamine groups may be distributed in the acetylated polyglucosamine in blocks such that there are blocks comprising at least 2, 3, 4, 5, 10, 15, 20, 40 or 50 β-(1→4)-linked D-glucosamine units (i.e. non-acetylated units) in a row.

The skilled person will recognise that the block pattern may not be uniform and blocks of varying lengths may be provided in a given acetylated polyglucosamine polymer and/or distribution of the block pattern may differ between different acetylated polyglucosamine polymers in a composition thereof. In one embodiment, the block length of the N-acetyl-D-glucosamine groups or the β-(1→4)-linked D-glucosamine units (i.e. non-acetylated units) may refer to the majority (e.g. over 50%) of the blockwise distribution. Alternatively, the block length of the N-acetyl-D-glucosamine groups or the β-(1→4)-linked D-glucosamine units (i.e. non-acetylated units) may refer to the average of the blockwise distribution, for example where some blocks of β-(1→4)-linked D-glucosamine units (i.e. non-acetylated units) are longer than others, and some blocks of β-(1→4)-linked D-glucosamine units (i.e. non-acetylated units) may be interrupted/separated with one or more N-acetyl-D-glucosamine groups.

In one embodiment according to the first aspect, the acetylated polyglucosamine comprises no more than 8% acetylation and the N-acetyl-D-glucosamine groups may be distributed in the acetylated polyglucosamine in blocks such that there are blocks comprising at least 2, 3, 4, 5, 10, 15, 20, 40 or 50 β-(1→4)-linked D-glucosamine units (i.e. non-acetylated units) in a row. In another embodiment according to the first aspect, the acetylated polyglucosamine comprises no more than 5% acetylation and the N-acetyl-D-glucosamine groups may be distributed in the acetylated polyglucosamine in blocks such that there are blocks comprising at least 2, 3, 4, 5, 10, 15, 20, 40 or 50 β-(1→4)-linked D-glucosamine units (i.e. non-acetylated units) in a row. In another embodiment according to the first aspect, the acetylated polyglucosamine comprises no more than 2% acetylation and the N-acetyl-D-glucosamine groups may be distributed in the acetylated polyglucosamine in blocks such that there are blocks comprising at least 2, 3, 4, 5, 10, 15, 20, 40 or 50 β-(1→4)-linked D-glucosamine units (i.e. non-acetylated units) in a row.

In one embodiment according to the second aspect, the acetylated polyglucosamine comprises no more than 90% acetylation and the N-acetyl-D-glucosamine groups may be distributed in the acetylated polyglucosamine in blocks such that there are blocks comprising at least 2, 3, 4, 5, 10, 15, 20, 40 or 50 β-(1→4)-linked D-glucosamine units (i.e. non-acetylated units) in a row. In another embodiment according to the second aspect, the acetylated polyglucosamine comprises no more than 20% acetylation and the N-acetyl-D-glucosamine groups may be distributed in the acetylated polyglucosamine in blocks such that there are blocks comprising at least 2, 3, 4, 5, 10, 15, 20, 40 or 50 β-(1→4)-linked D-glucosamine units (i.e. non-acetylated units) in a row. In another embodiment according to the second aspect, the acetylated polyglucosamine comprises no more than 50% acetylation and the N-acetyl-D-glucosamine groups may be distributed in the acetylated polyglucosamine in blocks such that there are blocks comprising at least 2, 3, 4, 5, 10, 15, 20, 40 or 50 β-(1→4)-linked D-glucosamine units (i.e. non-acetylated units) in a row. In another embodiment according to the second aspect, the acetylated polyglucosamine comprises no more than 80% acetylation and the N-acetyl-D-glucosamine groups may be distributed in the acetylated polyglucosamine in blocks such that there are blocks comprising at least 2, 3, 4, 5, 10, 15, 20, 40 or 50 β-(1→4)-linked D-glucosamine units (i.e. non-acetylated units) in a row. In another embodiment according to the second aspect, the acetylated polyglucosamine comprises no more than 90% acetylation and the N-acetyl-D-glucosamine groups may be distributed in the acetylated polyglucosamine in blocks such that there are blocks comprising at least 2, 3, 4, 5, 10, 15, 20, 40 or 50 β-(1→4)-linked D-glucosamine units (i.e. non-acetylated units) in a row. In another embodiment according to the second aspect, the acetylated polyglucosamine comprises no more than 95% acetylation and the N-acetyl-D-glucosamine groups may be distributed in the acetylated polyglucosamine in blocks such that there are blocks comprising at least 2, 3, 4, 5, 10, 15, 20, 40 or 50 β-(1→4)-linked D-glucosamine units (i.e. non-acetylated units) in a row.

In an embodiment comprising distributed blocks of β-(1→4)-linked D-glucosamine units, the block distribution may be for substantially all of the acetylated polyglucosamine molecules in a population of the acetylated polyglucosamine. The pattern of distribution and/or size of the blocks of β-(1→4)-linked D-glucosamine units may be heterogenous. In one embodiment, the acetyl groups are clustered in blocks across the polyglucosamine backbone (i.e. not evenly/homogenously distributed).

The polyglucosamine or the acetylated polyglucosamine may have a molecular weight of at least about 100 kDa. In another embodiment, the polyglucosamine or the acetylated polyglucosamine may have a molecular weight of at least about 115 kDa. In one embodiment, the polyglucosamine or the acetylated polyglucosamine may have a molecular weight of between about 100 kDa and about 500 kDa. In one embodiment, the polyglucosamine or the acetylated polyglucosamine may have a molecular weight of between about 100 kDa and about 1,000 kDa. In another embodiment, the polyglucosamine or the acetylated polyglucosamine may have a molecular weight of between about 115 kDa and about 1,000 kDa. Reference to the molecular weight of the polyglucosamine or the acetylated polyglucosamine is intended to refer to the average molecular weight of a composition of the polyglucosamine or the acetylated polyglucosamine polymers.

In one embodiment, a mixture of the polyglucosamine and the acetylated polyglucosamine may be administered/used.

In one embodiment, the adjuvant is administered without an antigen, such as a pre-defined and purified. The antigen may not be administered in combination with, concurrently or sequentially with the adjuvant. Instead, the adjuvant may trigger endogenous antigen release upon injection.

The Cancer

The cancer may be a solid tumour cancer. The solid tumour cancer may be selected from sarcoma, carcinoma, and lymphoma. In one embodiment, the solid tumour cancer comprises melanoma. In another embodiment, the solid tumour cancer comprises colon cancer. In another embodiment, the cancer comprises a cancer selected from the group comprising CNS tumor, breast cancer, leukemia, lung cancer, melanoma, prostate, AML, esophageal, ovarian, bladder, glioma, and multiple myeloma.

The Anti-Tumour Antigen

The anti-tumour antigen may comprise any known antigen for cancer, such as solid tumour cancer. The antigen for cancer may be selected from the group of antigens comprising TAA (tumour associated antigen) peptide mix, P10s-PADRE, PR1 peptide, Neo-antigen, MUC-1, pBCAR3 peptide, MAGE-A3, MAG-Tn3, PRAME, Neo-antigen, Bcl-XL, NY-ES0-1, WT1 peptide, GAA/TT-peptide, NY-ES0-1b, URLC10-177, MAGE-3.1, gp100, Pros.spec. peptide, ras peptide, THERATOPE, Globo H-GM2, sialylLewis^{a} (CA19-9), MUC-2-KLH, NY-ESO-1, CDX-1401, CDX-1307, Tumor lysate, CDX-1401, HER2 Peptide, ALVAC-NY-ESO-1, CMB305, SVN53-67-KLH, PR1 peptide, MART-1, and rsPSMA; or combinations thereof. Such suitable antigens for cancer are described in Schijns et al. (2020 Immunological Reviews, DOI: 10.1111/imr.12889), which is herein incorporated by reference. In one embodiment, the antigen for cancer is one or more tumour associated antigen(s). In one embodiment, the antigen for cancer is a neoantigen.

The neoantigen may be multiple antigens that are specific to the cancer of a patient. The skilled person will recognise that neoantigens are a subset of tumour specific antigens generated by non-synonymous mutations and other genetic variations specific to the genome of a tumour, presented by major histocompatibility complex (MHC) molecules, and recognized by endogenous T cells. The most commonly studied class of neoantigens are those derived from single-nucleotide variants (SNVs), which cause non-synonymous changes in a protein that subsequently may trigger antigen-specific T cell responses against the tumour. These can have an advantage over other classes of tumour antigens by having no expression in normal tissues. The neoantigens may be metastatic melanoma neoantigens. In another embodiment, the neoantigens may be neoantigens of lung cancer. In another embodiment, the neoantigens are of CNS tumor, breast cancer, leukemia, lung cancer, melanoma, prostate, AML, esophageal, ovarian, bladder, glioma, or multiple myeloma.

In one embodiment, antigens for ovarian cancer may comprise all of, or a selection of, the group of antigens comprising GM2, globo H, sTn, TF, and Ley. In one embodiment, antigens for prostate cancer may comprise all of, or a selection of, the group of antigens comprising GM2, Tn, sTn, TF, and Ley. In one embodiment, antigens for breast cancer may comprise all of, or a selection of, the group of antigens comprising GM2, globo H, Tn, sTn, TF, and Ley. In one embodiment, antigens for small-cell lung cancer may comprise all of, or a selection of, the group of antigens comprising GM2, fucosyl GM1, polysialic acid, globo H, and sialyl Lea. In one embodiment, antigens for sarcoma may comprise all of, or a selection of, the group of antigens comprising GM2, GD2, and GD3. In one embodiment, antigens for neuroblastoma may comprise all of, or a selection of, the group of antigens comprising, GM2, GD2, GD3, and polysialic acid. In one embodiment, antigens for melanoma may comprise all of, or a selection of, the group of antigens comprising GM2, GD2, and GD3.

In one embodiment, an antigen for a CNS tumour may comprise TAA peptide mix. In one embodiment, antigens for breast cancer may comprise one or more of the group of antigens comprising P10s-PADRE, MAG-Tn3, THERATOPE, and HER2 Peptide. In one embodiment, an antigen for leukemia may comprise PR1 peptide. In one embodiment, antigens for lung cancer may comprise one or more of the group of antigens comprising MUC-1, PRAME, NY-ESO-1b, ras peptide, and CMB305. In one embodiment, antigens for melanoma may comprise one or more of the group of antigens comprising pBCAR3 peptide, MAGE-A3, NY-ESO-1, MAGE-3.1, gp100, NY-ESO-1, CDX-1401, CDX-1401, and MART-1. In one embodiment, antigens for prostate cancer may comprise one or more of the group of antigens comprising Bcl-XL, Pros.spec. peptide, sialylLewis^{a} (CA19-9), MUC-2-KLH, and rsPSMA. In one embodiment, an antigen for AML may comprise WT1 peptide. In one embodiment, an antigen for esophageal cancer may comprise URLC10-177. In one embodiment, antigens for ovarian cancer may comprise one or more of the group of antigens comprising Globo H-GM2, and ALVAC-NY-ESO-1. In one embodiment, an antigen for bladder cancer may comprise CDX-1307. In one embodiment, an antigen for glioma may comprise GAA/TT-peptide and/or tumor lysate. In one embodiment, an antigen for multiple myeloma may comprise SVN53-67-KLH. In one embodiment, an antigen for a solid tumour may comprise Neo-antigen and/or ras peptide. In one embodiment, the antigen may comprise a tumour lysate, tumour extract or killed tumour cells.

The Combination and Dosage of the Anti-Tumour Antigen and Adjuvant

The skilled person will be familiar with antigen and adjuvant administration routes and doses. For example the administration may be sub-cutaneous, intra-muscular, intradermal, intraperitoneal or intravenous. According to the first aspect, the adjuvant and/or the anti-tumour antigen may be delivered, or arranged to be delivered, intravenously, subcutaneously, intradermally, intraperitoneally or intramuscularly. Preferably, the adjuvant and/or the anti-tumour antigen is delivered, or arranged to be delivered, subcutaneously or intramuscularly. In another embodiment according to the first aspect, the administration of the adjuvant and/or the anti-tumour antigen may intratumoural and/or peritumoural. In one embodiment, the administration of the adjuvant and/or antigen is sub-cutaneous, intra-muscular or intratumoral.

According to the first aspect, the adjuvant may be administered before, concurrently, or after the anti-tumour antigen(s). The adjuvant may be formulated separately from, or with, the anti-tumour antigen (i.e. to be delivered in the same dose or in separate doses).

The adjuvant and the anti-tumour antigen may be formulated together or separately in a pharmaceutically acceptable carrier.

A dose of the adjuvant and/or the anti-tumour antigen may be an amount sufficient to provide an immunogenic effect in the subject. In one embodiment, the dose of the adjuvant is sufficient to activate cGAS-STING dependent innate immune signalling in the tumour. The dose of the adjuvant may be sufficient to activate IFNAR-dependent Th1 responses and antigen-specific IgG2c production. A dose may be administered multiple times until the therapeutic/immunogenic effect is observed, such as the induction of a sufficient immune response in a subject. In one embodiment the administration of the anti-cancer antigen and adjuvant may comprise a prime-boost regime (i.e. a first dose followed by one or more further doses).

The anti-tumour antigen and adjuvant may be used as a vaccine in combination with another therapeutically or prophylactically active ingredient.

The other therapeutically or prophylactically active ingredient may comprise or consist of CpG (CpG oligodeoxynucleotides (or CpG ODN), which are short single-stranded synthetic DNA molecules). In another embodiment, the other therapeutically or prophylactically active ingredient may comprise or consist of other agonists of toll like receptors, nod-like receptors, C type lectin receptors, AIM 2 like receptors or other pathogen recognition receptors or a combination of pathogen recognition receptor agonists.

Intratumoral/Peritumoral Administration of Polyglucosamine or Chitosan into the

Solid Tumour According to the second aspect, the intratumoral/peritumoural administration of polyglucosamine or chitosan into the solid tumour of the subject may comprise the administration of a therapeutically effective amount of polyglucosamine or chitosan. The therapeutically effective amount may be sufficient to activate cGAS-STING dependent innate immune signalling in the tumour, and optionally activate IFNAR-dependent Th1 responses and antigen-specific IgG2c production.

The polyglucosamine or chitosan may be administered intratumorally and/or peritumourally by injection. In a preferred embodiment, the administration is intratumoural. However, peritumoural administration may be advantageous in cases of necrotic lesions in the centre of the tumour mass.

The polyglucosamine or chitosan may be administered in an amount sufficient to cause a therapeutic effect. The polyglucosamine or chitosan may be administered as a single dose, or multiple doses. Repeated doses may be administered until a therapeutic effect is observed, such as a reduction in tumour size, partial or complete cell death of the tumour cells. In an embodiment wherein the polyglucosamine or chitosan are repeatedly administered, the time between doses may be between 2 weeks and 4 weeks. Repeat administrations may be daily (e.g. for up to 1 week), sequential days, or every 3 days, for example for up to a month. Repeated administrations may be provided in a first week to a month time period, and then less regularly for a period of one or more months. In one embodiment repeated doses are provided about 1, 2 or 3 times per week. In another embodiment there is at least 1 week between doses. In another embodiment there is at least 2 weeks between doses.

The polyglucosamine or chitosan may be administered/used alone or administered/used in combination with another therapeutic. The polyglucosamine or chitosan may be administered/used in combination with a checkpoint inhibitor for the treatment of cancer. Therefore, in one embodiment, the method or use according to the invention may further comprise the administration of a checkpoint inhibitor.

The other therapeutically or prophylactically active ingredient may additionally or alternatively comprise or consist of CpG (CpG oligodeoxynucleotides (or CpG ODN), which are short single-stranded synthetic DNA molecules), MPL or another pathogen recognition receptor agonist or combination of pathogen recognition receptor agonists.

In one embodiment, the checkpoint inhibitor is formulated and administered separately from the polyglucosamine or chitosan. For example, polyglucosamine or chitosan may be administered intratumorally and the checkpoint inhibitor may be administered systemically.

The checkpoint inhibitor may be selected from anti-PD1 antibodies, anti-PDL1 antibodies, anti-CTLA-4 antibodies, anti-TIGIT antibodies and CD40 agonistic antibodies. In one embodiment, the checkpoint inhibitor may be selected from nivolumab, pembrolizumab, atezolizumab, avelumab, and tiragolumab. In one embodiment, the checkpoint inhibitor may be an antibody targeting the same protein or pathway as any of the antibodies selected from nivolumab, pembrolizumab, atezolizumab, avelumab, and tiragolumab. In one embodiment, the checkpoint inhibitor may be an antibody that competes for binding with any of the antibodies selected from nivolumab, pembrolizumab, atezolizumab, avelumab, and tiragolumab.

The polyglucosamine or chitosan may be administered/used in combination with an adjuvant. For example, an adjuvant that induces a CD8 response may be administered before, during or after administration of the polyglucosamine or chitosan. The polyglucosamine or chitosan may be formulated with the adjuvant.

The adjuvant may be an immunostimulant. The adjuvant may comprise an agonist of a pathogen recognition receptor that enhances cellular immunity, such as toll like receptor (TLR) agonists, for example CpG (TLR9), MPL (TLR4), NLR agonists, C-type lectin receptor (CLR) agonists, or AIM2-like receptor or RIG I agonists. The agonist may be a particulate adjuvant that promotes cellular immunity, such as nanoparticles, CAF01, IC31, AS01, attenuated viruses (e.g. measles vaccine) or attenuated bacteria (e.g. BCG vaccine).

The polyglucosamine or chitosan may be administered/used in combination with a cytokine, such as GM-CSF, IL-12, or TNF-α.

The polyglucosamine or chitosan may be provided in a carrier for the intratumoral/peritumoural administration. The carrier may be an aqueous carrier. In one embodiment, the carrier is a pharmaceutically acceptable carrier.

The Subject

The subject may be mammalian. In one embodiment, the subject is human. In another embodiment, the subject may be a domestic or livestock animal. The subject may be a subject with a solid tumour cancer, such as melanoma. The subject may have a sarcoma, carcinoma, or lymphoma.

Other Aspects

According to another aspect of the present invention, there is provided an adjuvant for use in combination with an anti-tumour antigen for immunotherapy for cancer in a subject, wherein the adjuvant comprises polyglucosamine or acetylated polyglucosamine that comprises no more than 10% N-acetyl-D-glucosamine groups, and wherein the N-acetyl-D-glucosamine groups are distributed in the acetylated polyglucosamine in blocks of two or more.

According to another aspect of the present invention, there is provided a composition comprising an adjuvant and an anti-tumour antigen, wherein the adjuvant comprises polyglucosamine or acetylated polyglucosamine that comprises no more than 10% N-acetyl-D-glucosamine groups, and wherein the N-acetyl-D-glucosamine groups are distributed in the acetylated polyglucosamine in blocks of two or more.

The composition may comprise a pharmaceutically acceptable carrier. The composition may be sterile/sterilised. The composition may be immunogenic, for example in a mammal, such as a human.

According to another aspect of the present invention, there is provided a kit comprising:
i) an adjuvant; and
ii) an anti-tumour antigen,
   wherein the adjuvant comprises polyglucosamine or acetylated polyglucosamine that comprises no more than 10% N-acetyl-D-glucosamine groups, and wherein the N-acetyl-D-glucosamine groups are distributed in the acetylated polyglucosamine in blocks of two or more.

The adjuvant and anti-tumour antigen of the kit may be formulated separately in a pharmaceutically acceptable carrier.

According to another aspect of the present invention, there is provided the use of polyglucosamine or chitosan in immunotherapy for solid-tumour cancer in a subject.

The polyglucosamine or chitosan may be used as an active therapeutic, for example as a standalone therapeutic or a therapeutic in combination with a checkpoint inhibitor. In one embodiment, the use is not as an adjuvant. The use of polyglucosamine or chitosan as a therapeutic may not be in combination with a vaccine or antigen, such as an anti-tumour vaccine/antigen.

The immunotherapy for solid-tumour cancer may comprise a step of intratumoral and/or peritumoural administration of the polyglucosamine or chitosan. In particular, the use in immunotherapy may be by intratumoral administration and/or peritumoural administration.

According to another aspect of the present invention, there is provided a composition comprising polyglucosamine or acetylated polyglucosamine, wherein the composition is formulated for intratumoral and/or peritumoural administration.

The acetylated polyglucosamine may comprise no more than 10% N-acetyl-D-glucosamine groups, and wherein the N-acetyl-D-glucosamine groups are distributed in the acetylated polyglucosamine in blocks of two or more.

The composition may comprise one or more pharmaceutically acceptable excipients.

Reference to “intratumoral administration” herein is intended to refer to direct injection into a solid tumour. Reference to “peritumoural administration” herein is intended to refer to direct injection into a space or tissue surrounding a solid tumour.

Reference to “solid tumour” herein is intended to refer to an abnormal mass of tissue or growth in a subject's body. Solid tumours may be benign (not cancer), or malignant (cancer). In one embodiment, the solid tumour is malignant.

Reference to “immunotherapy” herein is intended to refer to the treatment of disease by activating or suppressing the immune system. Immunotherapies designed to elicit or amplify an immune response are classified as activation immunotherapies, while immunotherapies that reduce or suppress are classified as suppression immunotherapies. The method or use of the invention herein for immunotherapy may be activation immunotherapy designed to elicit or amplify an immune response against the cancer cells or components thereof.

A Checkpoint inhibitor therapy is a form of cancer immunotherapy. A checkpoint inhibitor targets immune checkpoints, key regulators of the immune system that stimulate or inhibit its actions, which tumours can use to protect themselves from attacks by the immune system. Checkpoint therapy can block inhibitory checkpoints, restoring immune system function.

The term “immunogenic”, when applied to the present invention means capable of eliciting an immune response in a human or animal body. The immune response may be protective. The term “protective” means prevention of a cancer, a reduced risk of cancer, reduced cancer progression, reduced severity of cancer, a cure of a cancer, an alleviation of symptoms, or a reduction in severity of a cancer or cancer symptoms.

The term “treatment”, means a cure of cancer, an alleviation of symptoms, or a reduction in severity of a cancer or cancer symptoms.

Reference to “homogenous” distribution is understood to mean that the acetyl groups are distributed evenly across the polyglucosamine backbone. Reference to “heterogenous” distribution is understood to mean that the acetyl groups are clustered together.

The skilled person will understand that optional features of one embodiment or aspect of the invention may be applicable, where appropriate, to other embodiments or aspects of the invention.

Embodiments of the invention will now be described in more detail, by way of example only, with reference to the accompanying drawings.

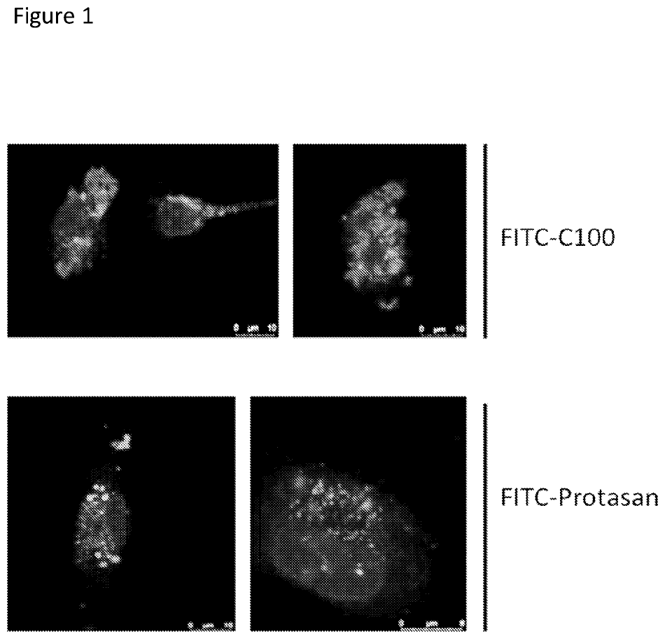

FIG. 1. BMDCs phagocytose C100 and Protasan. BMDCs were incubated with FITC-labelled C100 and Protasan CL213 (green) (5 μg/ml) for 6 h. Cells were subsequently washed, and mitochondria were stained with Mitotracker Red (red) and nuclei stained with Hoechst 33342 (blue). Cells were analysed by confocal microscopy. Results are representative of two independent experiments.

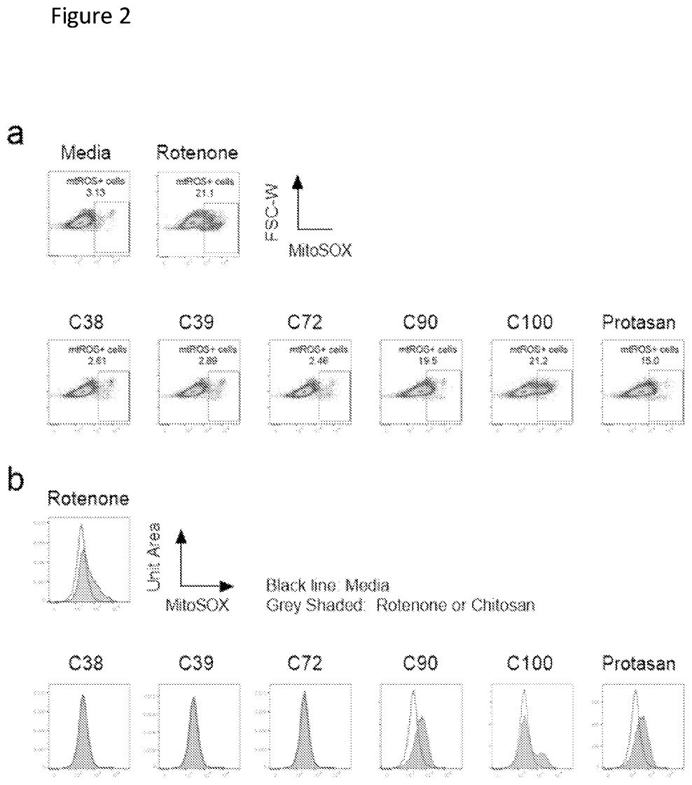

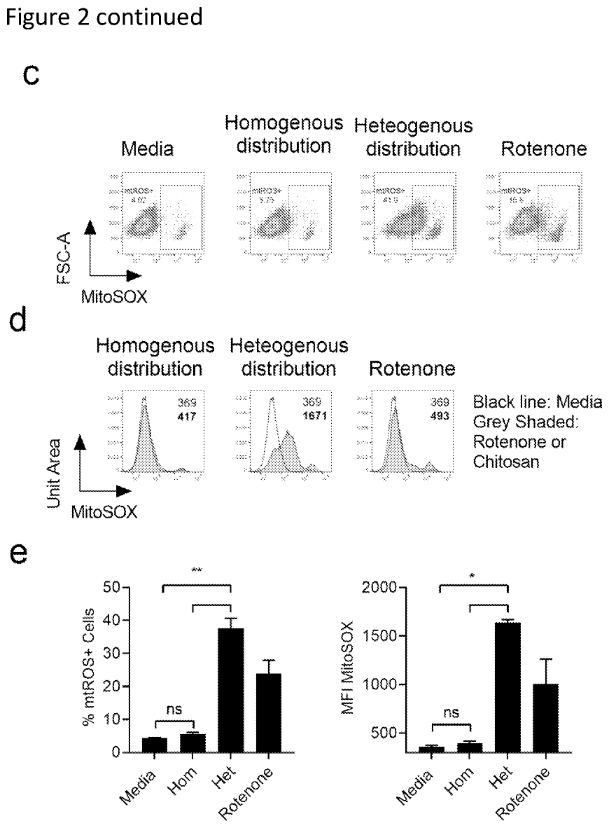

FIG. 2. Highly deacetylated chitosan polymers enhance production of mitochondrial reactive oxygen species.

(a-b) Analysis of mtROS production in single, live BMDCs treated with indicated concentrations of chitin-derived polymers (5 μg/mL) for 1.5 hours or rotenone (5 μM) for 6 hours. (a) FSC-W vs MitoSOX (b) Unit area vs MitoSOx. Data are representative of four independent experiments (c-e) Analysis of mtROS production in single, live BMDCs treated with homogenous and heterogenous 83% deactylated chitosan polymers (8 μg/mL) for 1.5 hours or rotenone (5 μM) for 6 hours. (c) FSC-W vs MitoSOX (d) Unit area vs MitoSOx. (e) Data combined from the two independent experiments were analysed by one-way ANOVA. *p<0.05**p<0.01. MFI=Mean fluorescence intensity.

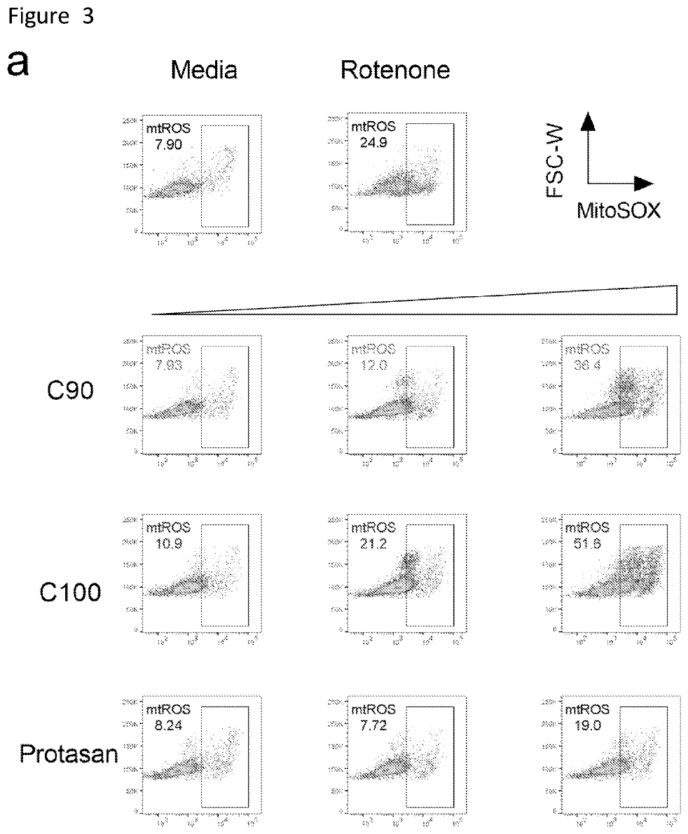

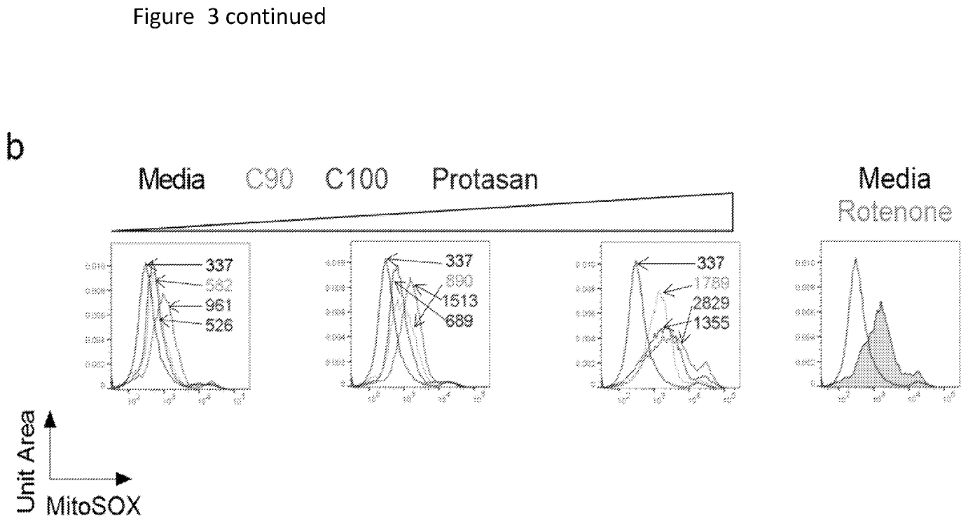

FIG. 3. Highly deacetylated chitosan polymers drive mitochondrial stress in BMDCs. (a-b) Analysis of mtROS production in single, live BMDCs treated with indicated concentrations of chitin-derived polymers (2,4 or 8 μg/mL) for 1.5 hours or rotenone (5 μM) for 6 hours. (a) FSC-W vs MitoSOX (b) Unit area vs MitoSOx. Values correspond to mean fluorescence intensity. Data is representative of four independent experiments.

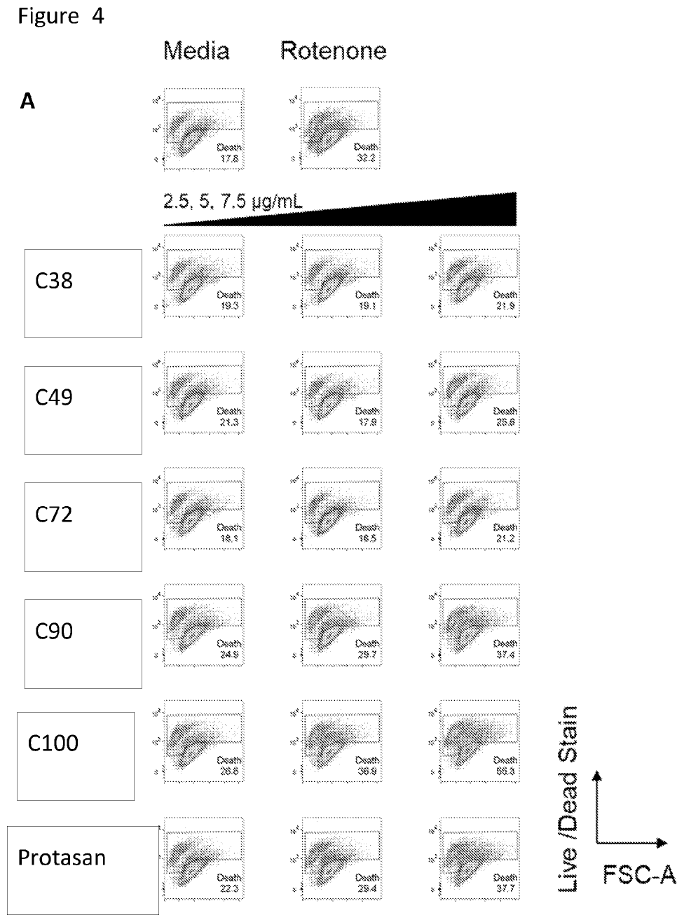

FIG. 4. Highly deacetylated chitosan polymers are toxic to BMDCs BMDCs were treated for 2 hours with chitin-derived polymers of various deacetylation (5 μg/mL) or Rotenone (5 μM) for 6 hours. (a) Flow cytometric analysis of cell viability. Data is representative of four independent experiments. MFI=Mean fluorescence intensity.

FIG. 5. The degree of polymer deacetylation dictates the extent of type I IFN production and IFNAR-dependent DC maturation. (a) BMDCs from WT and ifnar^{-/-} mice were treated with C90, C100 or protasan (2, 4 or 8 ug/mL), CpG (4 ug/mL) or LPS (10 ng/mL) for 24 hours. After this period, cells were collected and stained for flow cytometry. BMDCs were gated as single, live, CD11c+, MHCcII^{Hi} cells and then analysed for expression of CD80 and CD86. Figures show representative histograms and Mean fluorescence intensity values for CD86 and CD80. Data are representative of 3 independent experiments. (c) ELISA analysis of IFN-β secretion in the supernatants of BMDCs stimulated for 24 hours with indicated concentrations of homogenous and heterogenous chitosan polymers or CpG (4 ug/mL). Results are expressed as the mean±SD for technical triplicates and represent data from three independent experiments.

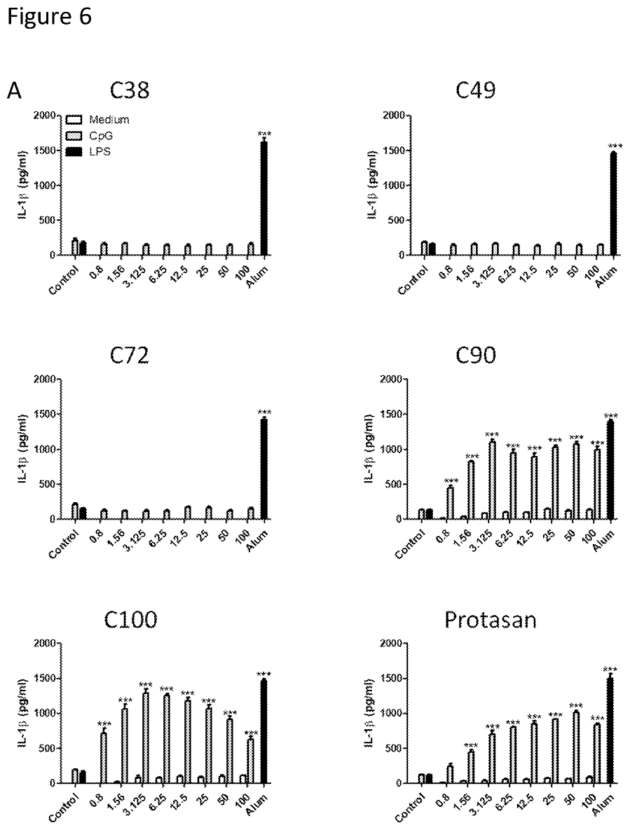

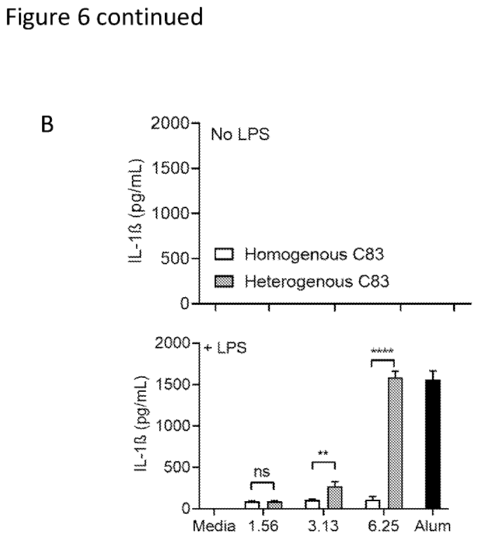

FIG. 6. Chitin-derived polymer-induced inflammasome activation by BMDCs is dependent on the degree and distribution of deactylation. (a) BMDCs were stimulated with medium, CpG (4 ug/mL) or LPS (10 ng/m) alone or together with chitin-derived polymers (0.8-6.25 ug/mL) or Alum (10 ng/mL). Levels of IL-1β were measured in supernatants 24 hours later. Results are expressed as the mean±SD for technical triplicates and are representative of three independent experiments. Statisitical analysis was performed by one way ANOVA with Dunnett's multiple comparisons test, CpG vs CpG+Chitin-derived polymer. ***p<0.001****p<0.0001. (b) BMDCs were stimulated with medium or LPS (10 ug/mL) alone or in combination with homogenous or heterogenous chitosan polymers (6.25 ug/mL) or alum (10 ug/mL). Levels of IL-1p were measured in supernatants 24 hours later. Results are expressed as the mean±SD for technical triplicates and are representative of three independent experiments. (a) Statisitical analysis was performed by one way ANOVA with Dunnett's multiple comparison test. (a) CpG vs CpG+Chitin-derived polymer. (b) LPS vs LPS+chitin-derived polymer. p****<0.0001.

FIG. 7. C100 induces mitochondrial ROS, DC maturation and Inflammasome activation across a broad range of molecular weights. (a) BMDCs were treated with chitin-derived polymers (8 ug/mL) for 1.5 hours or Rotenone (5 uM) for 6 hours. Single live cells were analysed for % of mtROS⁺ cells and MitoSOX fluourescense. Data combined from the two independent experiments was analysed by one-way ANOVA with Tukeys multiple comparison test. MFI=Mean fluorescence intensity. (b) BMDCs were treated with C90, C100 or protasan (2, 4 or 8 ug/mL), CpG (4 ug/mL) or LPS (10 ng/mL) for 24 hours. After this period, cells were collected and stained for flow cytometry. BMDCs were gated as single, live, CD11c+, MHCcII^{Hi} cells and then analysed for expression of CD80 and CD86. Figures show representative histograms and MFI values for CD86 and CD80. Data are representative of four independent experiments. (d) BMDCs were stimulated with medium or LPS (10 ug/mL) alone or in combination with chitin-derived polymers (1.56 ug/mL) or alum (10 ug/mL). Levels of IL-1β were measured in supernatants 24 hours later. Results are expressed as the mean±SD for technical triplicates and are representative of three independent experiments. Statisitical analysis was performed by two way ANOVA with Sidak's multiple comparison test.

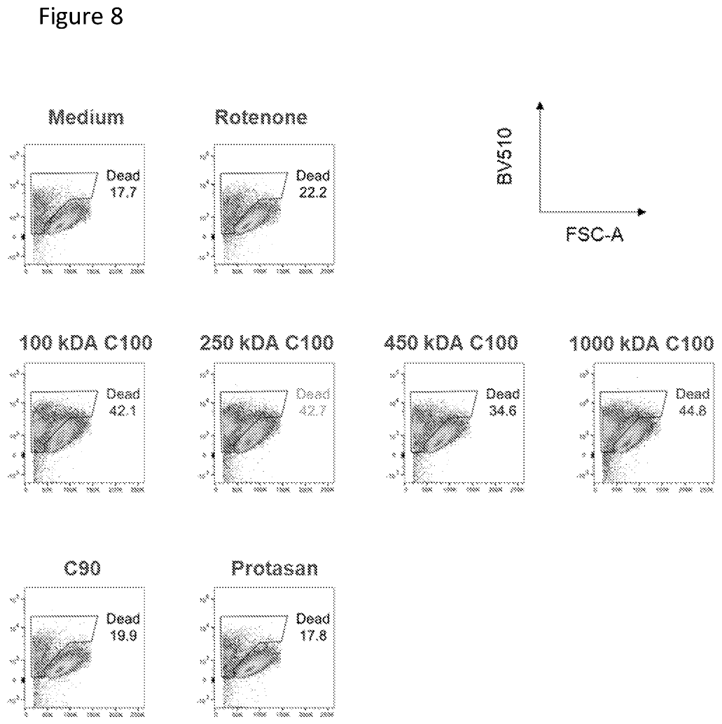

FIG. 8. C100 of various molecular weights are stronger inducers of mtROS and cell death than less deacylated chitosan polymers. BMDCs from WT mice were treated for 2 hours with 8 ug/mL of chitin-derived polymers or 6 hours with 1 uM Rotenone. Single, Live cells were analysed for MitoSOX fluorescence. (a) Gating strategy. (b) Percentage of mtROS+ cells in single live cells. (C) Histograms of MitoSOX fluorescence in single live cells. Values on graphs correspond to median fluorescence intensity (MFI) (d) Overlay histograms for polyglucosamine polymers of various molecular weights (e) Percentage cell death in single cells. Statistical analysis was determined by two-way ANOVA.

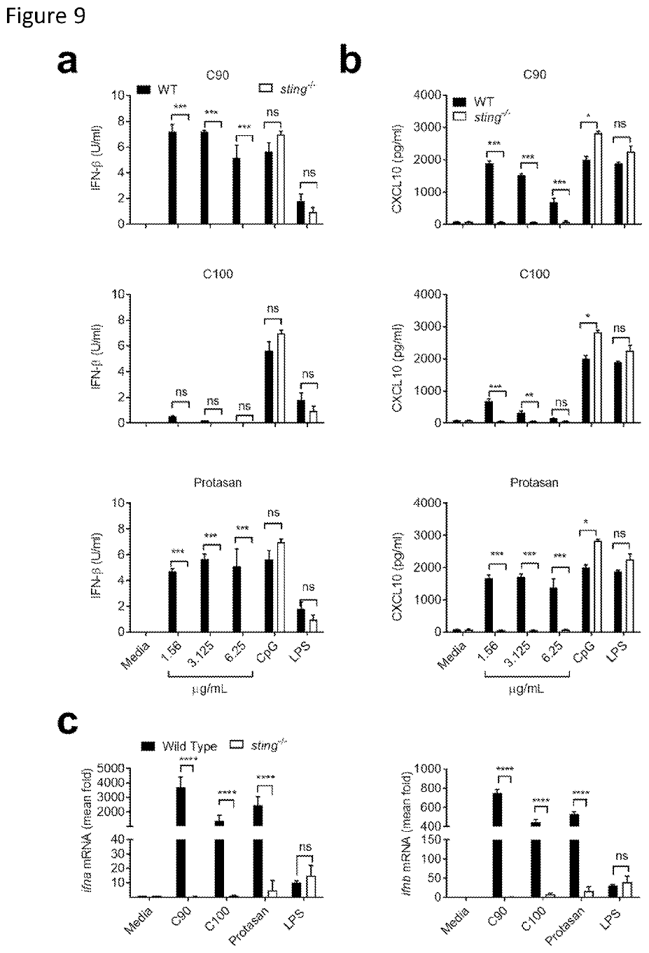

FIG. 9. Highly deactylated chitosan polymers are STING agonists. (a-b) ELISA analysis of (b) IFN-0 and (c) CXCL10 secretion in the supernatants of WT and sting^{-/-} BMDCs stimulated for 24 hours with indicated concentrations of chitin-derived polymers, CpG (4 ug/mL) or LPS (10 ng/mL). Results are expressed as the mean±SD for technical triplicates and represent data from three independent experiments. Statistical significance was determined by Two-tailed unpaired student's t tests with the Holm-sidak method for multiple comparisons were used ***p<0.001. (c) BMDCs from WT and sting^{-/-} mice were treated with chitin-derived polymers (8 μg/mL) for 24 hours or LPS (10 ng/mL) for 2 hours. mRNA levels were calculated by qPCR for ifna and ifnb with respect to β-actin and S18. Data shows technical triplicate mRNA levels with respect to b-actin and is representative of three independent experiments. Statistical significance was determined by Two-tailed unpaired student's t tests with the Holm-sidak method for multiple comparisons were used ****p<0.0001.

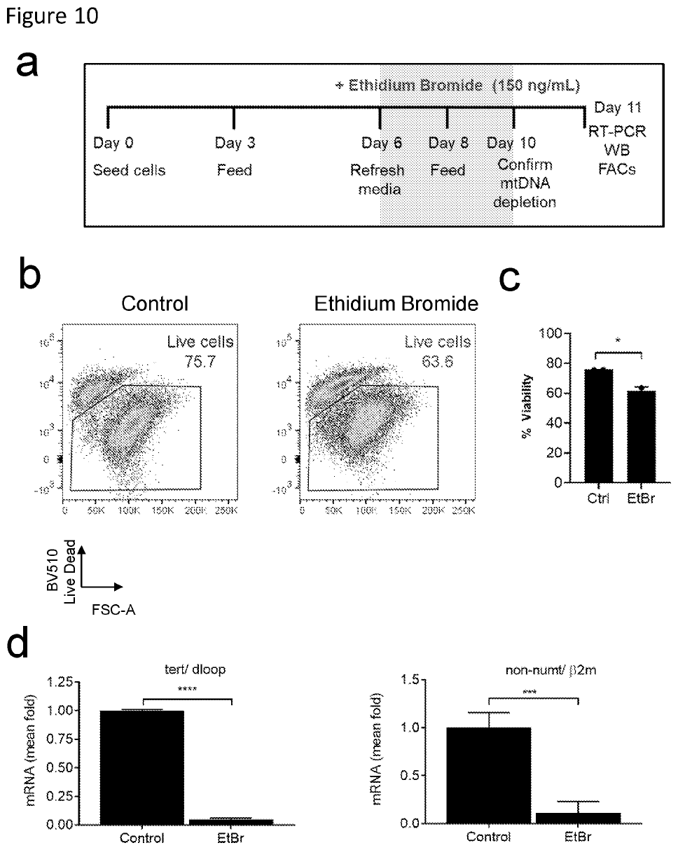

FIG. 10. Low dose ethidium bromide treatment depletes mtDNA in BMDCs.

(a) Experimental schematic used to deplete mitochondrial DNA (mtDNA) in BMDCs (see materials and methods for more details). (b) Day 11 flow cytometric analysis of ethidium bromide toxicity in single cells (data is representative of two independent experiments). (c) Toxicity data combined from two independent experiments was analysed by a two-tailed unpaired student's t test. *P<0.05. (d) on day 10, relative total mtDNA amounts were quantified by qPCR with primers specific for the mitochondrial D-loop region (dloop) or a region of mtDNA that is not inserted into nuclear DNA (non-NUMT) and primers specific for nuclear DNA (Tert, B2m). Statistical analysis on technical triplicates was performed by two-tailed unpaired student's t tests. Data are representative of four independent experiments. ***P<0.001; ****P<0.0001.

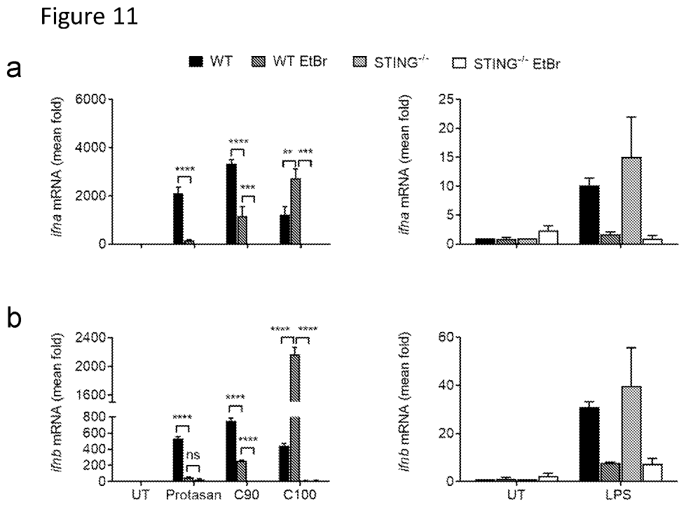

FIG. 11. C100-induced ifna and ifnb mRNA transcription does not require mtDNA.

Wild type (WT) and STING^{-/-} bone marrow precursors were cultured into BMDCs as normal (Control BMDCs) or in low dose ethidium bromide as explained in FIG. 10 to deplete mtDNA. Cells were then treated with chitin-derived polymers (5 μg/mL) for 24 hours or CpG (4 μg/mL) or LPS (10 ng/mL) for 3 hours. (a) Ifna and (b) ifnb mRNA levels were calculated by qPCR with respect to β-actin and S18. Results represent mRNA levels in technical triplicates with respect to β-actin and are representative of 5 independent experiments. Statistical analysis was performed by two-tailed unpaired student's t tests with the Holm-sidak method for multiple comparisons. **P<0.01; *** P<0.001; **** P<0.0001.

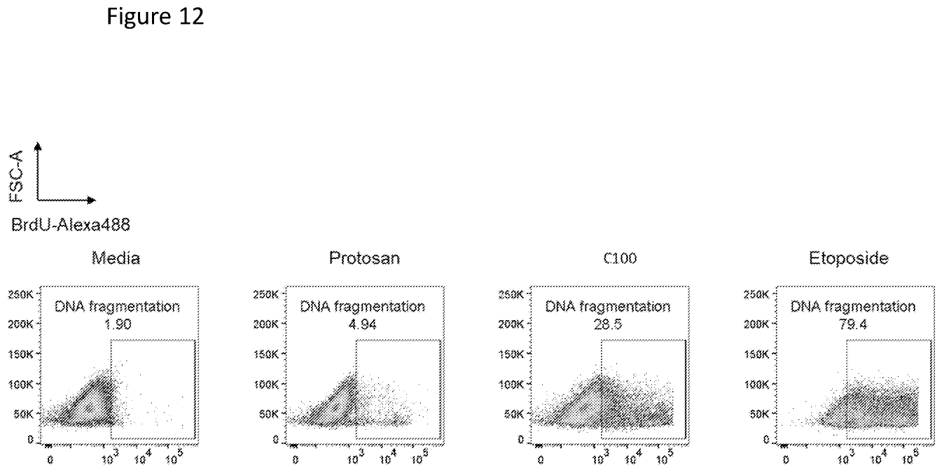

FIG. 12. C100 drives robust nuclear DNA fragmentation. BMDCs were treated for 9 hrs with 5 ug/mL of chitin-derived polymers. As controls cells were left untreated (media) or stimulated with 50 uM etoposide for 24 hrs. The BrdUTP/alex488-anti-BrdUTP TUNEL labelling system was used to monitor DNA fragmentation. Figure shows percentage of DNA fragmentation (BrdU-Alexa488+) as a percentage of total total DNA (PI+) in Single cells.

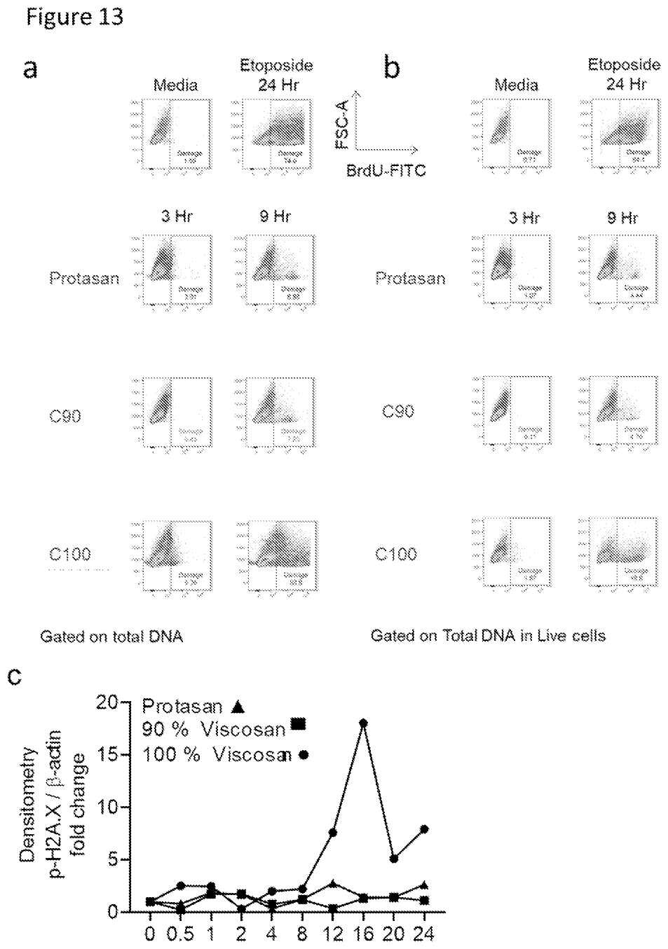

FIG. 13. Protasan and C90 do not cause DNA damage in BMDCs.

BMDCs were treated with 5 μg/mL of protosan, C90 or C100 for indicated times or 100 μM of etoposide for 24 hours. (a) DNA fragmentation as a percentage of total DNA in single cells (b) DNA fragmentation as a percentage of total DNA in single live cells. (c) BMDCs were stimulated for indicated times with C100, C90 or protasan and then lysed to determine expression levels of p-H2A.x. Densitomtrey readings of p-H2A.x expression levels with respect to b-actin compared to media treatment alone.

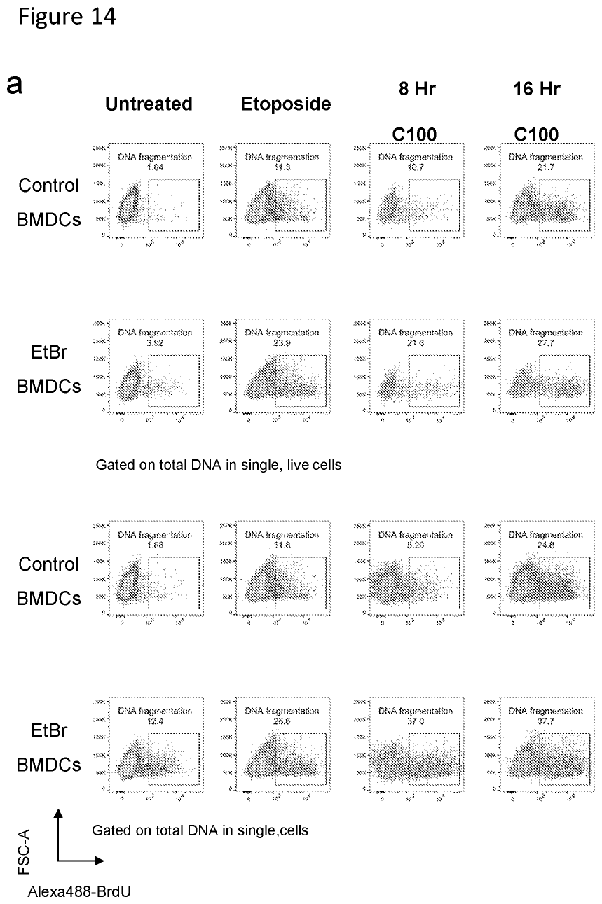

FIG. 14. Ethidium Bromide and C100 treatment drives synergistic DNA damage. (a) BMDCs were cultured as normal or in low dose ethidium bromide (see M&M for in-depth description of culture protocol) and then stimulated on day 10 with C100 (8 ug/mL) for 8 or 16 hours or with Etoposide (100 uM) for 16 hours. Cells were then lysed and blotted for expression of phosphorylated H2A.x. B-actin was used as a loading control. (b) Corresponding densitometry expression levels for p-H2A.x with respect to b-actin compared to the media treatment alone. Data is representative of two independent experiments. (c) BMDCs were cultured as normal (control BMDCs) or in low dose ethidium bromide (see M&M for indepth detail on culture protocol) and then stimulated on day 10 with C100 (8 ug/mL) for 8 or 16 hours or with Etoposide (100 uM) for 16 hours. Cells were collected and stained for analysis of DNA fragmentation by FACS. The degree of DNA fragmentation was measured as a percentage of the total DNA in (d) single, live cells and (e) single cells.

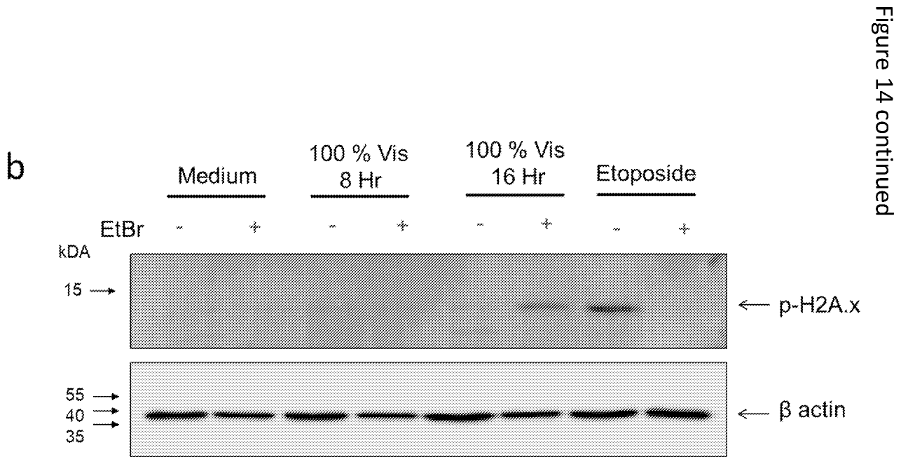

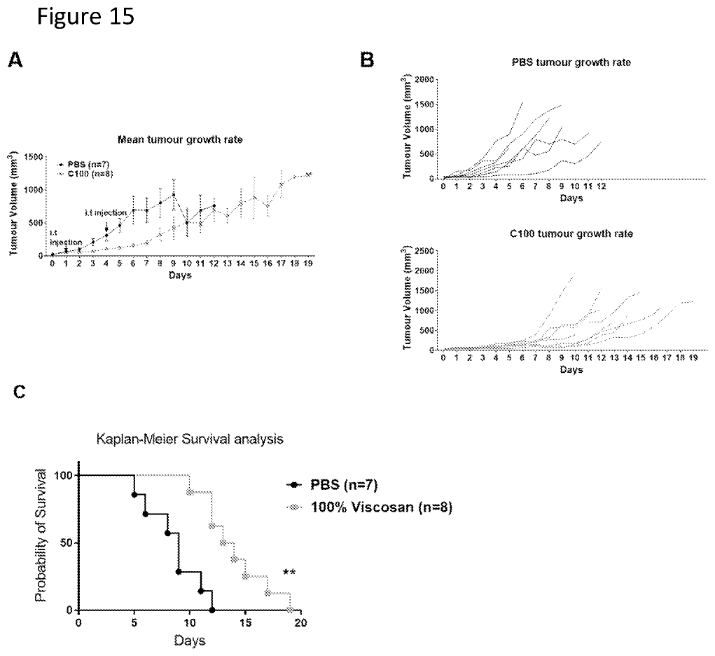

FIG. 15. Intratumoral delivery of C100 significantly inhibits tumour growth and promotes survival. Mice were injected s.c with 3.5×105 B16 melanoma cells. When tumours reached 75 mm3 they were injected with PBS or C100 (200 μg), repeated 3 days later at day 4. A) Tumour growth rate at challenge site displayed as mean tumour volume±SEM. A paired two tailed T test was used to determine statistical significance between PBS and C100 treated mice, where *p<0.05, **p<0.01, and ***p<0.001.) Spider plots of individual tumour growth. C) Kaplan-Meier survival analysis of challenged mice, a Mantel-Cox test was used to determine statistical significance between PBS and C100 treated mice where *p<0.05, **p<0.01, and ***p<0.001.

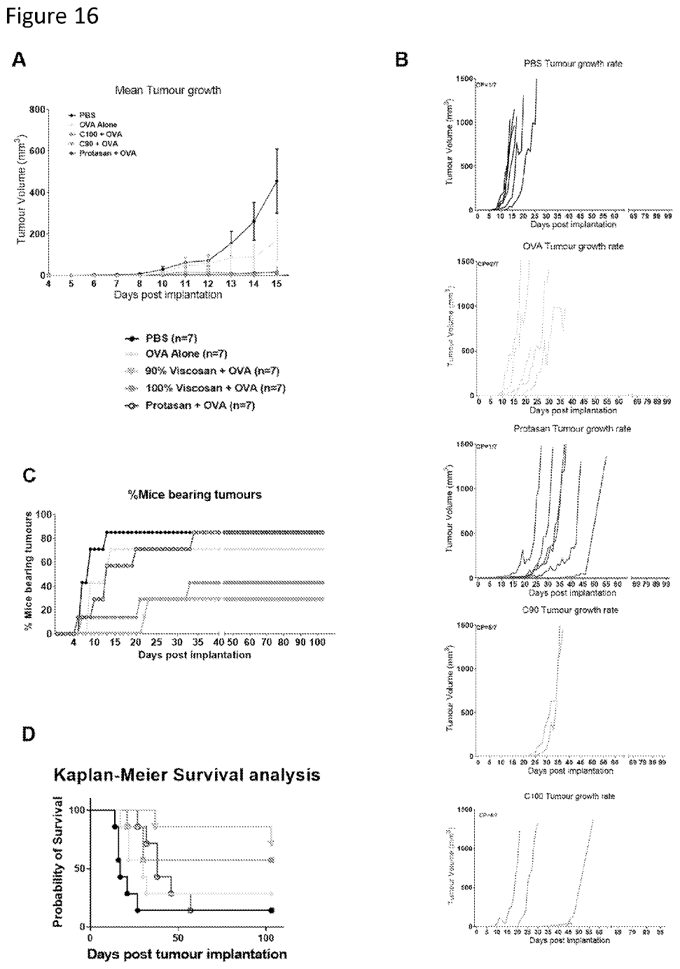

FIG. 16. C100 and C90 drive potent antigen specific anti-tumour immunity in a prophylactic setting

Mice were vaccinated on days 0 and 7 with PBS, OVA peptide (10 μg) alone, C100 (200 μg) plus OVA peptide (10 μg), C90 (200 μg) plus OVA peptide (10 μg), or Protasan (200 μg) plus OVA peptide (10 μg) followed by s.c challenge of 3.5×10⁵ B16-OVA tumour cells. A) Initial tumour growth rate at challenge site of mice bearing tumours prior to a humane endpoint, displayed as mean tumour volume±SEM. B) Spider plots of individual tumour growth. Proportions of mice displaying complete protection (CP) indicated on plots. C) Percentage of mice bearing tumours at challenge site post tumour implantation. C) Kaplan-Meier survival analysis of challenged mice.

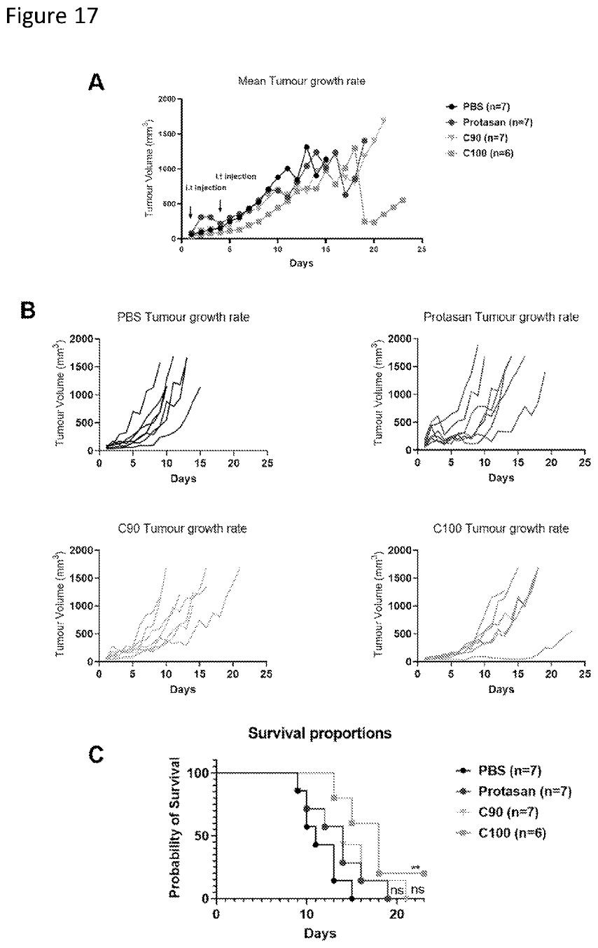

FIG. 17. Intratumoral delivery of C100, C90 and Protasan inhibits tumour growth and promotes extended survival. Mice were injected s.c with 3.5×105 B16 melanoma cells. When tumours reached 75 mm³ they were injected with PBS, C100 (200 μg), C90 (200 μg) or Protasan (200 μg), repeated 3 days later at day 4. A) Tumour growth rate at challenge site displayed as mean tumour volume. B) Spider plots of individual tumour growth. C) Kaplan-Meier survival analysis of challenged mice, a Mantel-Cox test was used to determine statistical significance between PBS and treated mice where ns=not significant, *p<0.05, **p<0.01, and ***p<0.001.

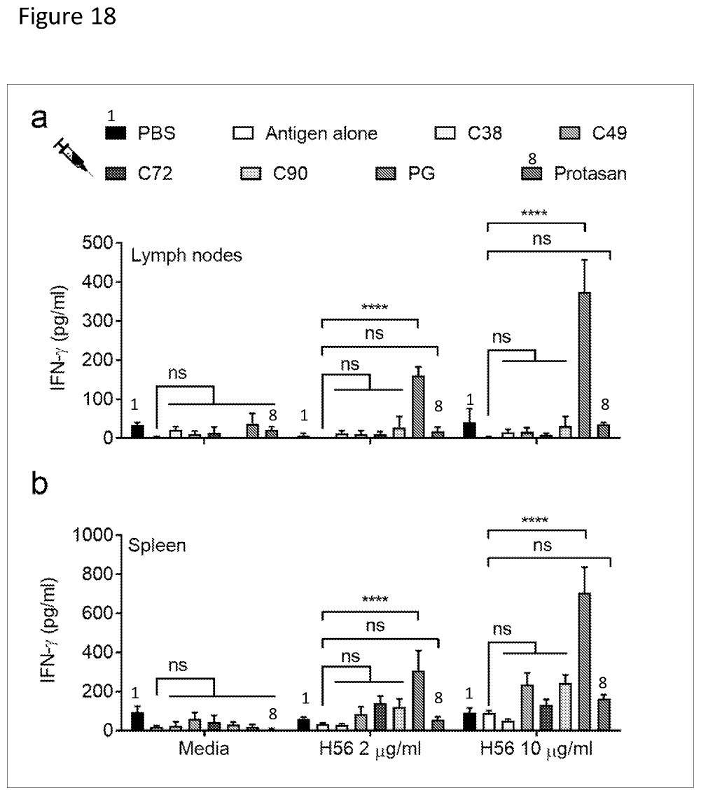

FIG. 18. Complete deacetylation of chitin is required to promote Th1 immune responses against the TB antigen H56 by the subcutaneous route C57BL/6 mice were immunised s.c on day 0 with PBS or H56 (0.5 μg), alone or in combination with chitin-derived polymers (200 μg/mouse). On day 14, mice were immunised with the same formulations and on day 21, spleens and inguinal lymph nodes were collected. (a) Splenocytes (2×10⁶ cells/mL) and (b) inguinal lymph nodes (1×10⁶ cells/mL) were restimulated ex-vivo with either media or H56 (2 μg/ml or 10 μg/ml). Levels of IFN-γ were measured in supernatants by ELISA after 72 hours. Data represent mean+SD for 5 mice per experimental group. Statistical analysis was determined by one-way ANOVA. Antigen alone Vs Antigen+Chitin-derived polymer. p****<0.0001.

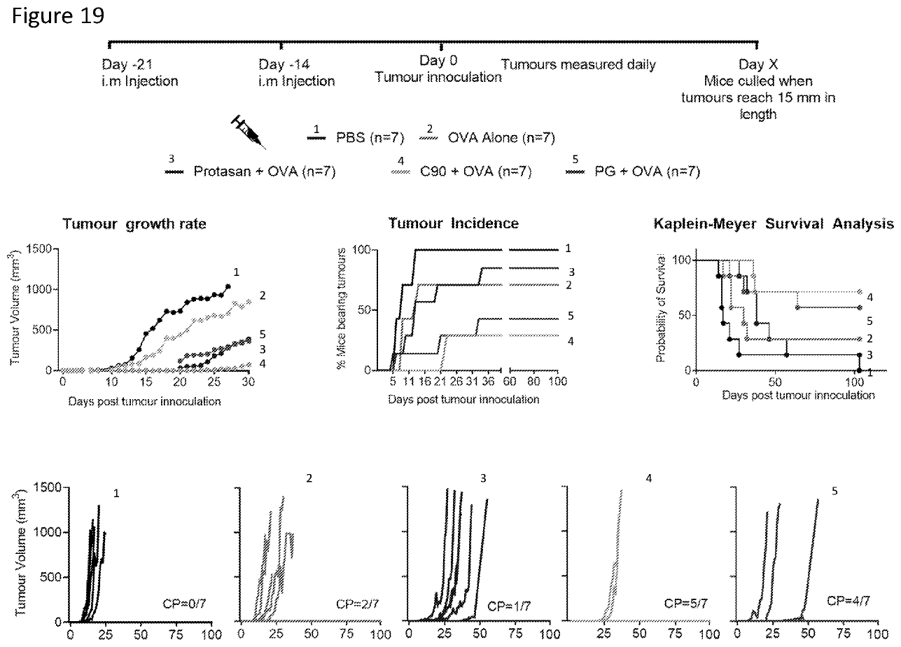

FIG. 19. Prophylactic immunisation with 86% deacetylated chitin and OVA does not reduce murine mortality against B16-OVA C57BL/6 mice were immunised i.m on day 0 with PBS or OVA alone or in combination with chitin-derived polymers (200 μg/mouse). On day 14, mice were inoculated s.c with 3.5×10⁵ B16 melanoma cells. (a) Schematic of experimental design. Tumour growth rate at challenge site displayed as mean tumour volume. An unpaired two tailed t test was used to determine statistical significance between PBS and chitosan treated mice, where *p<0.05, **p<0.01, and ***p<0.001. (c) Spider plots of individual tumour growth. (d) Kaplan-Meier survival analysis of challenged mice, a Mantel-Cox test was used to determine statistical significance between PBS and chitosan treated mice where **p<0.01.

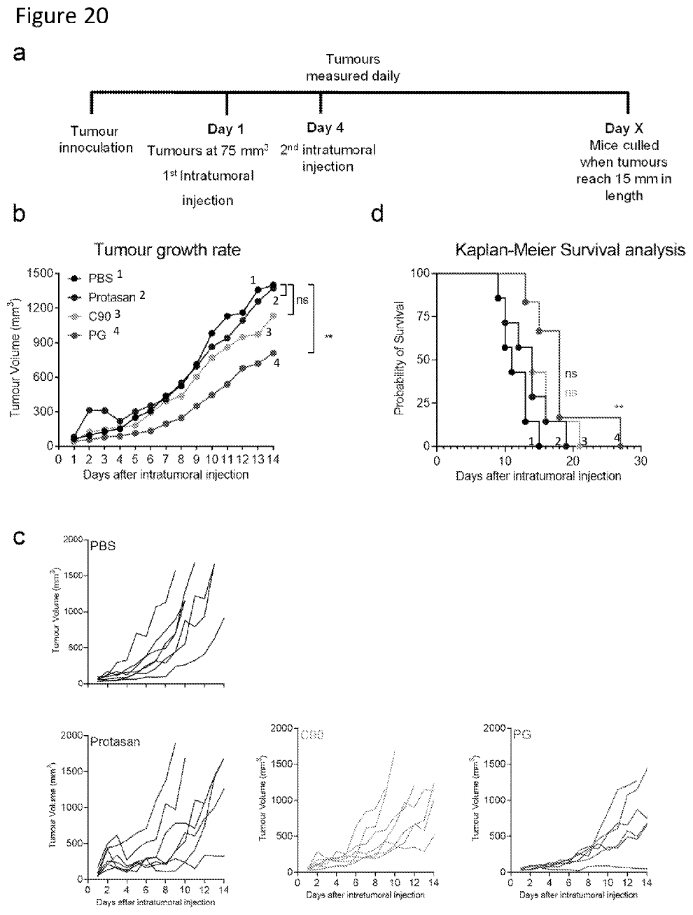

FIG. 20. Intratumoral delivery of 86% chitosan does not retard B16 tumour growth or extend mouse survival Mice were injected s.c with 3.5×10⁵ B16 melanoma cells. When tumours reached 75 mm³ they were injected intratumorally with 30 μL of PBS or chitin-derived polymers (200 μg). 3 days later tumours were injected as before. (a) Schematic of experimental design. (b) Tumour growth rate at challenge site displayed as mean tumour volume. An unpaired two tailed t test was used to determine statistical significance between PBS and chitosan treated mice, where *p<0.05, **p<0.01, and ***p<0.001. (c) Spider plots of individual tumour growth. (d) Kaplan-Meier survival analysis of challenged mice, a Mantel-Cox test was used to determine statistical significance between PBS and chitosan treated mice where **p<0.01.

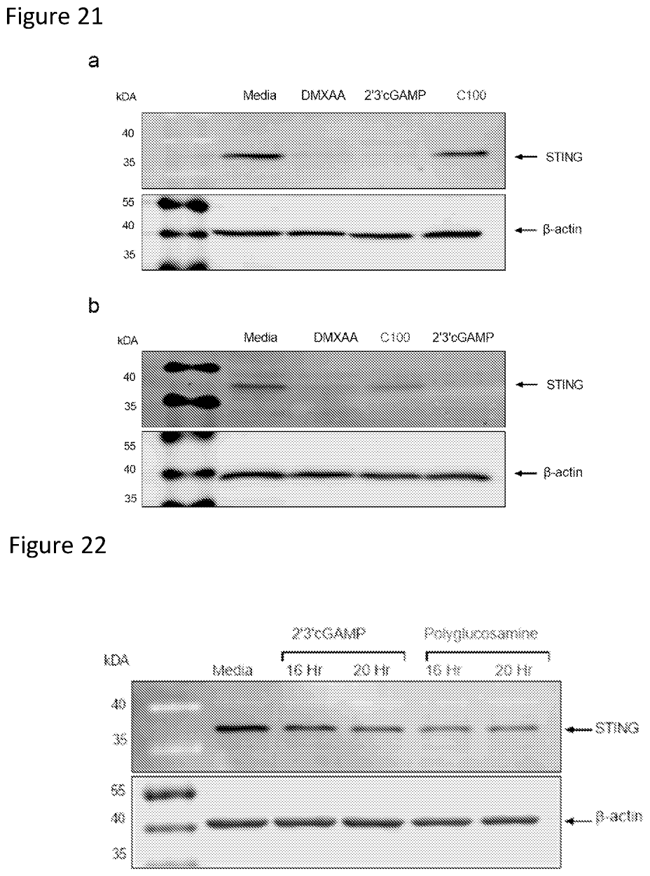

FIG. 21. C100 drives STING activation in B16 and MC38 tumour cell lines. (a) B16 and (b) MC38 (1×10\hat{ }6 cells/mL) were stimulated with DMXAA (10 ug/mL), C100 (10 ug/mL) or 2′3cGAMP (10 uM) for 20 hours. Cells were lysed and monitored for levels of STING and β-actin by immunoblot.

FIG. 22. C100 drives STING activation in human cells. (a) PMA-differentiated THP-1 cells (1×10\hat{ }6 cells/mL) were stimulated with C100 (10 ug/mL) or 2′3cGAMP (10 uM) for 16 or 20 hours. Cells were lysed and monitored for levels of STING and β-actin by immunoblot.

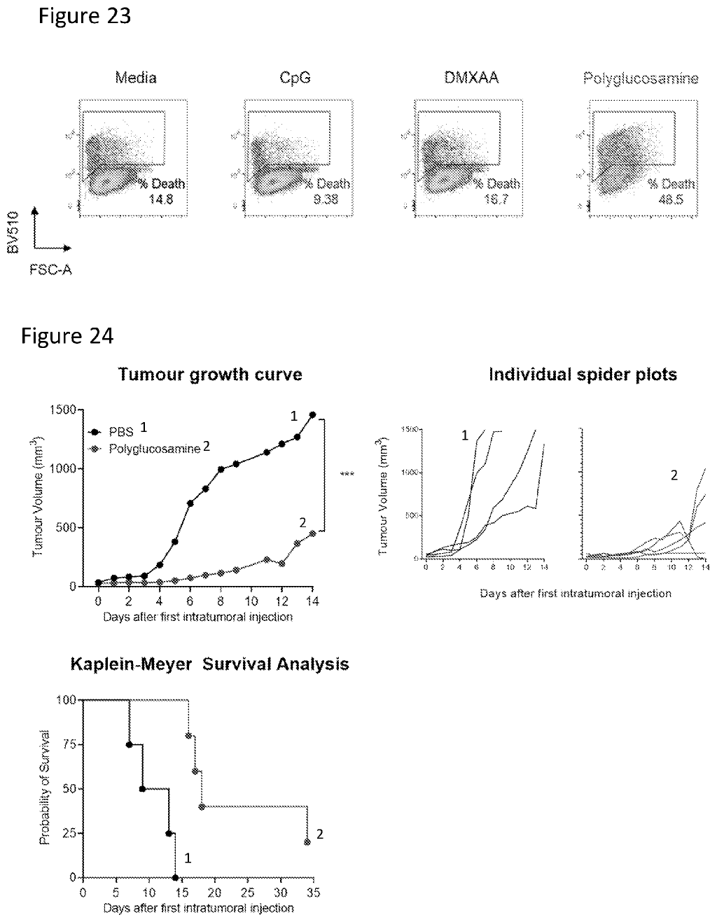

FIG. 23. C100 has tumoricidal activity (a) Percentage death in B16 cells (1×10\hat{ }6 cells/mL) stimulated with C100 (40 ug/mL), DMXAA (10 ug/mL) or CpG (4 ug/mL) for 20 hours.

FIG. 24. Intratumoral delivery of polyglucosamine retards MC38 tumour growth and extends mouse survival

Mice were injected s.c with 5×10⁵ B16 melanoma cells. When tumours reached 50 mm³ they were injected intratumorally with 30 μL of PBS or chitin-derived polymers (100 μg). 3 days later tumours were injected as before. Tumour growth rate at challenge site displayed as mean tumour volume. An unpaired two tailed t test was used to determine statistical significance between PBS and chitosan treated mice, where *p<0.05,**p<0.01, and ***p<0.001. Spider plots of individual tumour growth. Kaplan-Meier survival analysis of challenged mice, a Mantel-Cox test was used to determine statistical significance between PBS and chitosan treated mice where **p<0.01.

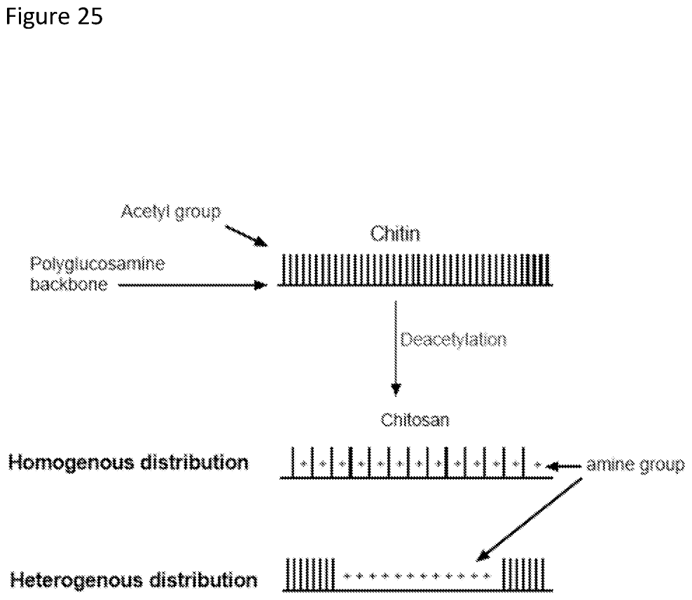

FIG. 25: Schematic diagram of homogenous acetyl group distribution compared to heterogenous acetyl group distribution in a polyglucosamine backbone. Homogenous distribution provides acetyl groups that are distributed evenly across the polyglucosamine backbone. Heterogenous distribution is understood to mean that the acetyl groups are clustered together.

Note: Polyglucosamine is sometimes referred to as 100% viscosan/C100 throughout the document.

### Example 1—Chitin Derived Polymer Deacetylation Regulates Reactive Oxygen Species Dependent cGAS-STING and NLRP3 Inflammasome Activation

### Summary

Chitosan is a cationic polysaccharide that has been evaluated as an adjuvant due to its biocompatible and biodegradable nature. The polysaccharide can enhance antibody responses and cell mediated immunity following vaccination by injection or mucosal routes. However, the optimal polymer characteristics for activation of dendritic cells (DCs) and induction of antigen-specific cellular immune responses have not been resolved. Here, we demonstrate that only polymers with a high degree of deacetylation enhance generation of mitochondrial ROS, leading to cGAS-STING induction of type I IFN. Additionally, the capacity of the polymers to activate the NLRP3 inflammasome were strictly dependent on the degree and pattern of deacetylation and mitochondrial ROS generation. Polymers with a degree of deacetylation (DDA) below 80% are poor adjuvants while a fully deacetylated ‘polyglucosamine’ polymer is most effective as a vaccine adjuvant. Furthermore, this polyglucosamine polymer enhanced antigen-specific Th1 responses in a NLRP3 and STING-type I IFN-dependent manner. Overall these results indicate that the degree of chitin derived polymer deacetylation and its regulation of mitochondrial ROS is the key determinant of its immune enhancing effects.

Materials and Methods:

Cell culture: Cells were cultured at 37° C. in an atmosphere maintained at 95% humidity and 5% CO₂.

Bone Marrow Derived Dendritic cells (BMDCs): Isolation and extraction: On day 1 C57BL/6 mice were sacrificed. The femurs and tibiae were removed, cleaned of muscle tissue with a sterile scissors and separated. Bones were washed in petri dishes containing 70% EtOH for 5 seconds and then cRPMI. A 10 mL syringe was filled and attached to a 27G needle. The tips of the femur and tibia bone were minimally cut and kept in a sterile petri dish with fresh cRPMI. The bone marrow was flushed out into a petri dish by inserting the 27G needle and injecting cRPMI. Cell clumps were disrupted by repeat gentle pipetting with a 19G needle. The cells were transferred to 50 mL falcon tubes and spun down at 1200 rpm for 5 minutes. The supernatant was removed and 1 mL of 0.88% NH₄Cl red blood cell lysis solution was added. After two minutes, the reaction was stopped with 25 mL of cRPMI. The cell mixture was centrifuged at 1200 rpm for 5 minutes. The supernatant was removed. The cell pellet was resuspended in 10 mL cRPMI. Cell numbers were counted using trypan blue dye exclusion (section 2.2.2.2). Cell suspension volume was adjusted with cRPMI containing 20 ng/mL GM-CSF to give a final concentration of 4.25×10⁵ cells/ml. Cells were transferred to T175 flasks (12.75×10⁶ cells/flask). From day 1 on, extreme care was taken when handling T175 flasks to avoid DC activation. On day 3, 30 mL of cDMEM containing 20 ng/mL GM-CSF was added to each T175 flask. On day 6 flask supernatants were removed and replaced with 30 mL fresh cRPMI containing 20 ng/mL GM-CSF. On Day 7, 30 mL of cRPMI containing 20 ng/mL GM-CSF was added to each T175 flask. On day 10 loosely adherent cells were removed by gentle repeat pipetting. Cells were transferred to 50 mL falcon tube and spun down at 1200 rpm for 5 minutes. The supernatant was discarded and the cell pellet was resuspended in 10 mL RPMI. Cell mixtures were made to a final concentration of 6.25×10⁵ cells/mL (unless stated otherwise) in cRPMI with 10 ng/mL GM-CSF. Cells were plated in round bottom 96 well plates at a volume of 200 μL/well for cytokine analysis or 12 well flat-bottom plates at a volume of 950 uL/well for FACs and qPCR analysis. The plates were incubated for at least 2 hours at 37° C. with 5% CO₂ before stimulation.

Measurement of Cytokine Secretion by ELISA

Concentrations of IL-6 were measured using antibodies obtained from BD Biosciences (BD 555240). IFN-β was detected using antibodies from Santa Cruz Biotechnology Inc. (Capture antibody #sc-57201) and R&D systems (detection antibody #32401-1). IFN-γ (DY485) CXCL10 (DY466) and IL-1β (DY401) ELISA kits were purchased from R&D systems.

Flow cytometric analysis of cell death: BMDCs were seeded at 1×10⁶ cells/mL in 12 well round flat bottom plates. After 24 hours of treatment with chitin-derivatives, plates were centrifuged and supernatants were discarded. Cells were removed from wells using ice cold PBS, transferred to labelled FACs tubes and washed with 2 mL PBS. Once washed and spun down, cells were stained with fixable viability stain 510 (400 μL: 1 in 1000 Dilution) or A700 in PBS for 15 minutes at room temperature. Cells were then washed twice in PBS and re-suspended in 200 uL FACS (2% Foetal calf serum (FCS) in PBS) buffer and acquired immediately and and the data analysed using Flowjo™ software (Treestar, Oregon).

Detection of Costimulatory Molecule Expression on Dendritic Cells

Cells were stained with Fixable Viability Stain 510, centrifuged at 400×g for 5 min at 4° C. and re-suspended in 100 μl of FACS buffer containing purified anti-mouse CD16/CD32 to block FcγRII/III. Cells were then surface stained by the addition of 10 μl of fluorochrome-conjugated antibodies against CD40, CD80 and CD86. Samples were acquired using a BD FACSCANTO II with summit software (Dako, Colorado) and the data analysed using Flowjo™ software (Treestar, Oregon).

Flow cytometric analysis of mitochondrial superoxide: BMDCs were seeded at 1×10⁶ cells/mL in 12 well round flat bottom plates. After 1.5 hour of treatment with chitin-derivatives, plates were centrifuged and supernatants were discarded. As a positive control for mitochondrial superoxide (mtROS), cells were treated with rotenone (5 μM) for 6 hours. Cells were removed from wells using ice cold PBS, transferred to labelled FACs tubes and washed with 2 mL PBS. Once washed and spun down, cells were co-stained with fixable viability stain 510 (200 μL: 1 in 1000 Dilution) and MitoSox Red (500 μL:1 in 5000 dilution) in PBS for 15 minutes at room temperature. Cells were then washed twice in PBS and re-suspended in 200 uL FACS buffer and acquired immediately and the data analysed using Flowjo™ software (Treestar, Oregon).

Flow cytometric analysis of DNA damage. The Apo-TUNNEL DNA damage kit was used to measure DNA fragmentation as follows—BMDCs were seeded at 1×10⁶ cells/mL in 12 well round flat bottom plates and treated with chitin-derivatives (5 μg/mL) for 3 or 9 hours. As a positive control, cells were treated with etoposide (100 uM) for 24 hours. Cells were removed from wells using ice cold PBS, transferred to labelled FACS tubes and washed with 2 mL of PBS. Cells were stained with A700 viability dye (400 uL; 1 in 1000 dilition) for 15 minutes at room temperature. After the elapsed time cells were washed and fixed in 1% PFA for 15 minutes. Cells were washed as before and then permeabilised overnight in pure ethanol at −20 C. The next morning, cells were washed and stained with the DNA labelling solution containing reaction buffer (10 uL/sample), TdT enzyme (0.75 uL/sample), BrdUTP (8 uL/sample) and dH₂O (31.25 uL/sample) for 1 hour at 37° C. Note, FACS tubes were shaken every 15 minutes to keep cells in suspension. After 1 hour, cells were washed with Rinse buffer (provided in kit) and stained with 100 uL of the antibody staining solution (10 uL Alexa 488 anti-BrdU Ab and 90 uL rinse buffer) for 30 minutes at room temperature. Without washing, 200 uL PI/RNase A staining buffer was added to each FACS tube already containing the antibody staining solution. Cells were analysed 30 minutes—2 hours after the addition of the PI/RNase A buffer on FACSCanto and the data analysed using Flowjo™ software (Treestar, Oregon).

Flow cytometric analysis of costimulatory molecule expression on B16-F10 cells: 2×10⁵ B16 melanoma cells were stimulated with media or 40 μg/ml, C90, C100 or protasan for 24 hours in 24 well plates. Cells were removed from wells using ice cold PBS-EDTA, transferred to labelled FACS tubes and washed with 2 mL of PBS. Cells were stained with BV510 viability dye (1 in 1000 dilition) for 15 minutes at room temperature. Cells were then washed in PBS, and stained for cell surface markers for 30 minutes at room temperature. Cells were then washed in PBS, resuspended in 200 d of FACs buffer, with samples acquired via BD FACSCanto and the data analysed using Flowjo™ software (Treestar, Oregon).

Quantitative Polymerase Chain Reaction:

Q-PCR

RNA was isolated using High Pure RNA Isolation Kit (Roche) and reverse transcribed into complementary DNA (cDNA) with an M-MLV reverse transcriptase, RNase H minus, point mutant (Promega). Quantitative PCR was performed using KAPA SYBR FAST qPCR Kit Master Mix (2×) (KAPA Bio-systems) in accordance with the instructions provided by the manufacturer using Aligent Technologies Stratagene Mx3005P technology. The following primers were used (5′→3′). Actb forward, TCCAGCCTTTCTTGGT, Actb reverse, GCACTGTGTTGGCATAGAGGTC, S18 forward, S18 reverse, Ifnb forward, ATGGTGGTCCGA GCAGAGAT, Ifnb reverse, CCACCACTCATTCTGAGGCA, Ifna forward, ATGGCTAGGCTCTGTGCTTTCCT, Ifna reverse, AGGGCTCTCCAGAGTTCT GCTCTG. RNA expression was normalized to β-actin and s18 expression and to expression in the relevant untreated control sample.

Depletion of mitochondrial DNA in BMDCs: Cells were cultured as normal until day 6 when media was replaced with 30 mL of RPMI containing 20 ng/mL of GM-CSF and 150 ng/mL of ethidium bromide. On day 7, a further 30 mL of RPMI containing 20 ng/mL of GM-CSF and 150 ng/mL of ethidium bromide was added to T175 flasks. On day 10, loosely adherent cells were removed by gentle pipetting, washed and resuspended in RPMI containing 10 ng/mL GM-CSF. 5×10\hat{ }6 cells were taken to confirm mtDNA depletion and the rest were plated for stimulations.

Quantification of mitochondrial DNA depletion: Total DNA was isolated from 5×10\hat{ }6 cells according to DNeasy Blood and tissue kit culture cells protocol. Mitochondrial depletion was quantified by qPCR with primers specific for the mitochondrial D-loop region (dloop) or a region of mtDNA that is not inserted into nuclear DNA (non-NUMT) and primers specific for nuclear DNA (Tert, B2m).

Western Blot:

Sample Preparation

BMDCs were seeded at 1×10⁶ cells/mL in 12 well plates and left to rest for a minimum of two hours before stimulation. Cells were lysed in 150 uL Lamelli Buffer containing 1% Protease inhibitor ( 1/100 dilution) 1% phosphatase inhibitor ( 1/100 dilution) and left on ice for 5 minutes. After 5 minutes, the lamelli-cell mixture was transferred to Eppendorf tubes and heated for 5 minutes at 95° C. in a heating block.

Sodium dodecyl sulfate (SDS) polyacrylamide gel electrophoresis SDS polyacrylamide gels were made. Isopropanol was used to removed bubbles from the lower gel and was poured out prior to addition of the stacking gel. Gels were placed in SDS electrophoresis stands with plates faced inwards. The SDS stand containing the two gels was then placed in the SDS machine and the centre column was filled to the top with SDS running buffer. The central SDS stand was checked for leaks. Combs were removed from the two gels to reveal lanes for protein loading. The samples (20 uL) and protein ladder (7 uL+13 uL lamelli buffer) were added into lanes. Any remaining empty lanes were filled with 20 uL lamelli buffer to ensure proteins travelled at an equal rate across the gel. Once fully loaded the outer part of the SDS machine was filled with running buffer to above the silver electrode line. The machines were then attached to powerpacks and ran at 80 Volts for 30 minutes (or until the lamelli buffer has passed through the stacking gel). The voltage was then increased to 100 V and left to run until the smallest protein band (10 kDa) was 5 mm away from the bottom of the plate.

Protein Transfer: Semi-Dry Method.

The two gels were removed from the central SDS stand. The upper plate was removed carefully exposing the gel. The gel was quickly submerged in transfer buffer. Two pieces of 1.5 mm filter paper were cut, each slightly buffer than the gel and soaked in transfer buffer. PVDF was cut to the same size of the gels and then activated in methanol for 2 minutes, followed by two 2-minute washes in water and transfer buffer. One piece of soaked filter paper was placed on the transfer machine. The activated PVDF membrane was then placed on top of the filter paper. The gel was carefully removed from transfer buffer and placed on top of the PVDF membrane. The second soaked filter paper was then placed on top of the gel. A roller was used to remove any air bubbles, taking care not to move the gel off the PVDF membrane. The gel was transferred using mAmps, with the number of mAmps dependent on the size of the protein of interest. For example, a 40 kDA protein was transferred using 50 mAmps per gel for 1 hr 30 minutes. If two gels were transferred at the same time, the machine was set to 100 mAMPs for 1 hr 30 minutes. Note: Do not exceed four gels per transfer machine. Once finished PVDF membranes were placed in 50 mL tubes with the side of the membrane in contact with the gel facing into the centre of the tube.

Blocking

Proteins were blocked in 10 mLs of 3% BSA in TBST for 1 hour at RT or overnight at 4° C. Note put 50 mL tubes on rotators to ensure even membrane blocking.

Capture Antibody

Blocking buffer was removed from the 50 mL tubes and replaced with 3 mL Primary antibody solution. Tubes were placed on rotators overnight at 4° C.

Secondary Antibody

The primary antibody mixture was poured out and membrane were washed three times in TBST for 5 minutes each. Gels were then placed in secondary antibody solutions for 1 hour at RT on rotators. After the hour, gels were washed three times in TBST for 5 minutes each. When the protein of interest was not phosphorylated, membranes were additionally washed twice in PBS for 5 minutes. Blots were analysed on an Odyssey LI-COR machine with the intensity set at 5 for the 700 channel and 6 for the 800 channel and resolution set to 169 μm. Images were analysed using Image studio Lite software.

Prophylactic tumour model: 10 week old C57BL/6J mice were given a prime boost vaccine regime involving intramuscular immunisations on days 0 and 14 with PBS, OVA peptide alone (10 μg), C100 (200 μg)+OVA peptide (10 μg), C90 (200 μg)+OVA peptide (10 μg), or Protasan (200 μg)+OVA peptide (10 μg). B16-F10 cells constitutively expressing OVA (B16-F10-OVA) were cultured in vitro utilising T175 cell culture flasks. Upon reaching logarithmic phase of growth (≤50% confluency) cells were harvested using Trypsin EDTA. Cells were washed, counted and resuspended to appropriate concentration in ice cold HBSS. Mice were subsequently challenged subcutaneously on the right flank with 3.5×10⁵ B 16-F10-OVA cells. Tumour growth was measured daily and tumour volume was determine using the formula: Volume=(Length×(Width)²)/2. Mice were culled at a humane endpoint determined as reaching 15 mm in diameter.

Therapeutic tumour model: B16-F10-OVA cells were cultured in vitro utilising T175 cell culture flasks. Upon reaching logarithmic phase of growth (≤50% confluency) cells were harvested using Trypsin EDTA. Cells were washed, counted and resuspended to appropriate concentration in ice cold HBSS. 12-week-old C57BL/6J mice were challenged with 3.5×10⁵ B16-F10 cells on the right flank. Tumour growth was measured daily and tumour volume was determined as (Length×(Width)²)/2. Upon tumour volume reaching 75 mm³, mice were randomly allocated to experimental groups with blocking, and received PBS or C100 (200 μg) intratumorally. This same treatment was repeated 3 days later. Tumour volume was subsequently measured daily as volume determined as before. Mice were culled at a humane endpoint determined as reaching 15 mm in diameter.

TABLE 1
The effect of C100, C90 and Protasan on costimulatory/co-inhibitory molecule
expression 2 × 10⁵ B16 melanoma cells were stimulated with media, C90 (40 μg/ml),
C100 (40 μg/ml), protasan (40 μg/ml), CpG (4 μg/ml), Etoposide (200 μM) or
DMXAA (10 μg/ml) for 24 hours and costimulatory/co-inhibitory molecule
expression were analysed via cell surface staining and acquisition on the BD
FACSCanto/BD LSR Fortessa. Results are represented as upregulated (√)
or no change (—) and as fold increase in MFI compared to unstimulated control.

| | Upregulated (√)/No change (—) | | | | | | |
|---|---|---|---|---|---|---|---|
| | Costimulatory | | | | Coinhibitory | | |
| | MHC CI | CD40 | CD80 | CD86 | TIGIT | PD-L1 | PD-1 |
| Unstimulated | — | — | — | — | — | — | — |
| C100 | √ √ | √ | √ | √ √ √ | √ | √ | √ |
| C90 | √ | — | — | — | √ | √ | — |
| Protasan | √ | — | — | — | √ | √ | — |
| CpG | — | — | — | — | — | √ | — |
| DMXAA | √ | — | — | — | √ | √ | √ |
| Etoposide | √ √ √ | √ √ | √ √ | √ √ | √ √ | √ | — |
| | Fold increase in MFI | | | | | | |
| | Costimulatory | | | | Coinhibitory | | |
| | MHC CI | CD40 | CD80 | CD86 | TIGIT | PD-L1 | PD-1 |
| Unstimulated | 1 | 1 | 1 | 1 | 1 | 1 | 1 |
| C100 | 2.0 | 1.6 | 1.3 | 2.8 | 1.6 | 1.3 | 1.1 |
| C90 | 1.3 | 1.1 | 0.9 | 1.1 | 1.5 | 1.2 | 1.0 |
| Protasan | 1.3 | 1.1 | 1.0 | 1.2 | 1.5 | 1.3 | 0.9 |
| CpG | 1.0 | 1.1 | 1.1 | 1.1 | 1.1 | 1.3 | 1.0 |
| DMXAA | 1.2 | 1.1 | 1.0 | 1.1 | 1.3 | 1.2 | 1.2 |
| Etoposide | 3.0 | 1.9 | 2.2 | 1.9 | 1.7 | 1.6 | 1.0 |

1. A method of immunotherapy for prevention or treatment of cancer in a subject, the method comprising the administration of an adjuvant, or administration of an adjuvant in combination with a tumor antigen, to the subject, wherein the adjuvant comprises polyglucosamine or acetylated polyglucosamine that comprises no more than 10% Nacetyl-D-glucosamine groups, and wherein the N-acetyl-D-glucosamine groups are distributed in the acetylated polyglucosamine in blocks of two or more.

2. The method according to claim 1, wherein the N-acetyl-D-glucosamine groups are distributed in the acetylated polyglucosamine in blocks of ten or more.

3. The method according to claim 1, wherein the tumor antigen is selected from the group of antigens consisting of: TAA (tumor associated antigen) peptide mix, P 10s-PADRE, PR1 peptide, Neo-antigen, MUC-1, pBCAR3 peptide, MAGE-A3, MAG-Tn3, PRAME, Neo-antigen, Bel-XL, NY-ESO-1, WT1 peptide, GAA/TT-peptide, NY-ESOlb, URLC10-177, MAGE-3.1, gp100, Pros.spec. peptide, ras peptide, THERATOPE, Globo H-GM2, sialylLewisa (CA19-9), MUC-2-KLH, NY-ESO-1, CDX-1401, CDX-1307, Tumor lysate, CDX-1401, HER2 Peptide, ALVAC-NY-ESO-1, CMB305, IDC-0305, and rsPSMA; and combinations thereof.

4. The method according to claim 1, wherein the tumor antigen comprises neoantigen.

5. The method according to claim 1, wherein the tumor antigen comprises a tumor lysate, tumor extract or killed tumor cells.

6. The method according to claim 1, wherein the anti-tumor antigen and adjuvant are used as a vaccine in combination with another therapeutically or prophylactically active ingredient;
optionally wherein the therapeutically or prophylactically active ingredient comprises CpG, agonists of toll like receptors, nod-like receptors, C type lectin receptors, AIM 2 like receptors or other pathogen recognition receptors or a combination of pathogen recognition receptor agonists.

7. A method of immunotherapy for solid-tumor cancer in a subject, the method comprising the administration of polyglucosamine or chitosan into the subject, wherein the administration is intratumoral and/or peritumoral.

8. The method according to claim 7, wherein the polyglucosamine or the acetylated polyglucosamine has a molecular weight of at least 100 kDa.

9. The method according to claim 7, wherein the acetylated polyglucosamine is chitosan and is at least 10% de-acetylated or comprises no more than 90% N-acetyl-D-glucosamine groups.

10. The method according to claim 7, wherein the N-acetyl-D-glucosamine groups are distributed in the acetylated polyglucosamine in blocks of two or more.

11. The method according to claim 7, wherein the N-acetyl-D-glucosamine groups are distributed in the acetylated polyglucosamine in blocks of ten or more.

12. The method according to claim 7, wherein the polyglucosamine or the acetylated polyglucosamine has a molecular weight of at least 100 kDa.

13. The method according to claim 7, wherein the polyglucosamine or chitosan is administered in combination with another therapeutic;
optionally wherein the therapeutic is a checkpoint inhibitor for the treatment of cancer; and/or
optionally wherein the therapeutic comprises of CpG, MPL or another pathogen recognition receptor agonist or combination of pathogen recognition receptor agonists.

14. The method according to claim 12, wherein the checkpoint inhibitor is selected from anti-PD1 antibody, anti-PDL1 antibody, anti-CTLA-4 antibody, anti-TIGIT antibody and CD40 agonistic antibody, nivolumab, pembrolizumab, atezolizumab, avelumab, and tiragolumab, or an antibody targeting the same protein or pathway as, or competing for binding with, any of the antibodies selected from nivolumab, pembrolizumab, atezolizumab, avelumab, and tiragolumab.

15. The method according to claim 7, wherein the polyglucosamine or chitosan is administered in combination with an adjuvant and/or a cytokine.

16. (canceled)

17. A composition comprising an adjuvant and an anti-tumor antigen, wherein the adjuvant comprises polyglucosamine or acetylated polyglucosamine that comprises no more than 10% N-acetyl-D-glucosamine groups, and wherein the N-acetyl-D-glucosamine groups are distributed in the acetylated polyglucosamine in blocks of two or more.

18. A kit comprising:
i) an adjuvant; and
ii) an anti-tumor antigen,
wherein the adjuvant comprises polyglucosamine or acetylated polyglucosamine that comprises no more than 10% N-acetyl-D-glucosamine groups, and wherein the N-acetyl-D-glucosamine groups are distributed in the acetylated polyglucosamine in blocks of two or more.

19. A method of immunotherapy for prevention or treatment of a solid tumor cancer in a subject, the method comprising: administering a therapeutically effective amount of polyglucosamine or chitosan to the subject.

20. The method according to claim 19, wherein the immunotherapy comprises a step of intratumoral and/or peritumoural administration of the polyglucosamine or chitosan.

21. The method according to claim 19 wherein the polyglucosamine or chitosan is not administered as an adjuvant and/or the not administered in combination with a vaccine or antigen.

22. A composition comprising polyglucosamine or acetylated polyglucosamine, wherein the composition is formulated for intratumoral and/or peritumoural administration.

23. The composition according to claim 22, wherein the acetylated polyglucosamine comprises no more than 10% N-acetyl-D-glucosamine groups, and wherein the N-acetyl-D-glucosamine groups are distributed in the acetylated polyglucosamine in blocks of two or more.

24. The composition according to claim 22, further comprising a checkpoint inhibitor and/or a therapeutic comprising one or more of CpG, MPL or another pathogen recognition receptor agonist or combination of pathogen recognition receptor agonists.

25. A kit comprising the composition according to claim 22, and a checkpoint inhibitor and/or a therapeutic comprising one or more of CpG, MPL or another pathogen recognition receptor agonist or combination of pathogen recognition receptor agonists.
